(19) United States
(12) Patent Application Publication
(10) Pub. No.: US 2026/0159575 A1
(43) Pub. Date: Jun. 11, 2026

(54) ANTI-TAU ANTIBODY COMPOSITIONS, DOSAGE FORMS, AND METHODS

(71) Applicant: EISAI R&D MANAGEMENT CO. LTD., Tokyo (JP)

(72) Inventors: Larisa REYDERMAN, Hackensack, NJ (US); Jin ZHOU, Short Hills, NJ (US); Kristin Ruth WILDSMITH, Mendham, NJ (US); Pau Aceves BALDO, Hatfield Herts (GB); Sumit RAWAL, Wilton, CT (US)

(21) Appl. No.: 18/706,852

(22) PCT Filed: Nov. 3, 2022
(86) PCT No.: PCT/IB2022/060604

§ 371 Date: May 2, 2024

Related U.S. Application Data
(60) Provisional application No. 63/275,045, filed on Nov. 3, 2021.
(60) Provisional application No. 63/290,278, filed on Dec. 16, 2021.
(60) Provisional application No. 63/316,582, filed on Mar. 4, 2022.
(60) Provisional application No. 63/316,616, filed on Mar. 4, 2022.

(51) Int. Cl. C07K 16/18; A61K 9/00; A61K 39/00; A61P 25/28

(52) U.S. Cl. CPC C07K 16/18; A61K 9/0019; A61P 25/28; A61K 2039/505; A61K 2039/545; C07K 2317/565; C07K 2317/76

(57) ABSTRACT

Provided herein are dosage forms comprising an antibody that specifically binds Tau, methods of treating a human subject diagnosed with a Tauopathy comprising administering an antibody that specifically binds Tau to the human subject, and pharmaceutical compositions for treating a subject diagnosed with a Tauopathy comprising an antibody that specifically binds Tau.

### CROSS-REFERENCE TO RELATED APPLICATIONS

This application claims the benefit of U.S. Application No. 63/275,045, filed Nov. 3, 2021, U.S. Application No. 63/290,278, filed Dec. 16, 2021, U.S. Application No. 63/316,582, filed Mar. 4, 2022, and U.S. Application No. 63/316,616, filed Mar. 4, 2022. Each of these applications is incorporated herein by reference in its entirety.

### SEQUENCE LISTING

The instant application contains a Sequence Listing which is being submitted herewith electronically in ASCII format and is hereby incorporated by reference in its entirety. Said ASCII copy, created on Oct. 26, 2022, is named 104018001201_SEQUENCE LISTING.xml and is 17,345 bytes in size.

### TECHNICAL FIELD

The present invention relates to dosage forms comprising an antibody that specifically binds Tau, to methods of treating a human subject diagnosed with a Tauopathy comprising administering to the subject an antibody that specifically binds Tau, and to pharmaceutical compositions for treating a human subject diagnosed with a Tauopathy comprising an antibody that specifically binds Tau.

### BACKGROUND

Tau proteins, which belong to the family of microtubule-associated proteins (MAPs), are mainly expressed in neurons and found in the axons and dendrites. Tau proteins play an important role in the assembly of tubulin monomers into microtubules to constitute the cytoskeleton and serve as tracks for axonal transport. Tau proteins are translated from a single gene located on chromosome 17, with alternative mRNA splicing leading to the formation of 6 different central nervous system tau isoforms, of which 5 are found in the human adult brain. The isoforms differ, having either 3 (R1, R3, and R4) or 4 (R1-R4) repeat-regions in the carboxy (C)-terminal part and variable occurrence of microtubule binding region (MTBR). The amino (N)-terminal domain, which establishes links between microtubules and other parts of the cytoskeleton, or the plasma membrane, has a variable occurrence of 0, 1, or 2 inserts of 29 amino acids.

Tau proteins are the key constituent of intracellular fibrillary tangles described in Alzheimer's disease (AD) and other neurodegenerative disorders, referred to as tauopathies. Aggregation of hyperphosphorylated tau into insoluble paired helical filaments (PHF) that accumulate in nerve cells is a critical process in the formation of neurofibrillary tangles (NFTs), which is a hallmark pathological finding in AD, a secondary tauopathy. NFTs are also a pathological finding in primary tauopathies, such as Frontotemporal Dementia, Corticobasal Degeneration, Pick's disease, and Progressive Supranuclear Palsy. Recent in vitro and in vivo research on the development of PHF and NFTs has shown that the pathophysiological tau process appears to be initiated by the occurrence of extracellular tau seeds. These small soluble tau seeds containing MTBR trigger the spread of tau pathology across the brain, possibly in a trans synaptic manner, inducing the formation of intracellular insoluble tau aggregates, thereby driving the development of NFT pathology. In AD, NFTs occur in a neuroanatomically characteristic pattern of increasing severity, generally defined according to the Braak stages 1 to 6, which correlate well with progressive neuronal loss and clinical decline. Consequently, selective targeting and removal of tau seeds is expected to stop or slow down disease progression in tauopathy or AD-associated tauopathy. At this time, however, there is no cure for and no way of slowing down the progression of these diseases. Thus, there is an urgent unmet medical need for drugs that slow or prevent the progression of tauopathies.

### SUMMARY

It is an object of the present invention to provide dosage forms comprising an antibody that specifically binds Tau, methods of treating a human subject diagnosed with a Tauopathy comprising administering to the subject an antibody that specifically binds Tau, and pharmaceutical compositions for treating a human subject diagnosed with a Tauopathy comprising an antibody that specifically binds Tau.

Provided herein are intravenous dosage forms comprising an antibody that specifically binds Tau, wherein the amount of antibody in a single dose is about 3 mg/kg to about 90 mg/kg. According to some embodiments, intravenous dosage forms comprising an antibody that specifically binds Tau, wherein the amount of antibody in a single dose is about 3 mg/kg, about 10 mg/kg, about 30 mg/kg, about 60 mg/kg or about 90 mg/kg, are provided.

Provided herein are intravenous dosage forms comprising an amount of an antibody that specifically binds Tau, wherein the amount is a single dose to achieve a serum Cₘₐₓ in the range of 6.29 μg/mL to 1960 μg/mL after administration to a human subject. Further provided herein are intravenous dosage forms comprising an antibody that specifically binds Tau, wherein the amount of the antibody is a single dose to achieve a geometric mean serum Cₘₐₓ of from about 9.55 μg/mL to about 1450 μg/mL after administration to a human subject.

Further provided herein are intravenous dosage forms comprising an amount of an antibody that specifically binds Tau, wherein the amount is a single dose to achieve a serum AUC_{(0-inf)} in the range of 12300 μg*hr/mL to 194000 μg*hr/mL after administration to a human subject. Further provided herein are intravenous dosage forms comprising an amount of an antibody that specifically binds Tau, wherein the amount is a single dose to achieve a geometric mean serum AUC_{(0-inf)} of from about 12300 μg*hr/mL to about 130000 μg*hr/mL after administration to a human subject.

Further provided herein are intravenous dosage forms comprising an amount of an antibody that specifically binds Tau, wherein the amount is a single dose to achieve a serum AUC₍₀₋₆₇₂ₕ₎ in the range of 839 μg*hr/mL to 203000 μg*hr/mL after administration to a human subject. Further provided herein are intravenous dosage forms comprising an amount of an antibody that specifically binds Tau, wherein the amount is a single dose to achieve a geometric mean serum AUC₍₀₋₆₇₂ₕ₎ of from about 1580 μg*hr/mL to about 122000 μg*hr/mL after administration to a human subject.

Provided herein are intravenous dosage forms comprising an amount of an antibody that specifically binds Tau, wherein the amount is a single dose to achieve a CSF Cₘₐₓ in the range of 13.5 ng/mL to 672 ng/mL after administration to a human subject. Further provided herein are intravenous dosage forms comprising an amount of an antibody that specifically binds Tau, wherein the amount is a single dose to achieve a geometric mean CSF Cₘₐₓ of from about 15.9 ng/mL to about 404 ng/mL after administration to a human subject.

Further provided herein are intravenous dosage forms comprising an amount of an antibody that specifically binds Tau, wherein the amount is a single dose to achieve a CSF AUC₍₀₋₂₄ₕ₎ in the range of 159 ng*hr/mL to 7690 ng*hr/mL after administration to a human subject. Further provided herein are intravenous dosage forms comprising an amount of an antibody that specifically binds Tau, wherein the amount is a single dose to achieve a geometric mean CSF AUC₍₀₋₂₄ₕ₎ of from about 191 ng*hr/mL to about 5320 ng*hr/mL after administration to a human subject.

Also provided herein are methods of treating a human subject diagnosed with a Tauopathy, comprising administering intravenously to the human subject an amount of an antibody that specifically binds Tau, wherein the amount is a single dose of about 3 mg/kg to about 90 mg/kg. According to some embodiments of the methods, the amount is a single dose of about 3 mg/kg, about 10 mg/kg, about 30 mg/kg, about 60 mg/kg, or about 90 mg/kg.

Also provided herein are methods of treating a human subject diagnosed with a Tauopathy, comprising administering intravenously to the human subject an amount of an antibody that specifically binds Tau, wherein the amount is a single dose to achieve a serum Cₘₐₓ in the range of 6.29 μg/mL to 1960 μg/mL after administration. Also provided herein are methods of treating a human subject diagnosed with a Tauopathy, comprising administering intravenously to the human subject an amount of an antibody that specifically binds Tau, wherein the amount is a single dose to achieve a geometric mean serum Cₘₐₓ of from about 9.55 μg/mL to about 1450 μg/mL after administration.

Also provided herein are methods of treating a human subject diagnosed with a Tauopathy, comprising administering intravenously to the human subject an amount of an antibody that specifically binds Tau, wherein the amount is a single dose to achieve a serum AUC_{(0-inf)} in the range of 12300 μg*hr/mL to 194000 μg*hr/mL after administration. Also provided herein are methods of treating a human subject diagnosed with a Tauopathy, comprising administering intravenously to the human subject an amount of an antibody that specifically binds Tau, wherein the amount is a single dose to achieve a geometric mean serum AUC_{(0-inf)} of from about 12300 μg*hr/mL to about 130000 μg*hr/mL after administration.

Further provided herein are methods of treating a human subject diagnosed with a Tauopathy, comprising administering intravenously to the human subject an amount of an antibody that specifically binds Tau, wherein the amount is a single dose to achieve a serum AUC₍₀₋₆₇₂ₕ₎ in the range of 839 μg*hr/mL to 203000 μg*hr/mL after administration. Further provided herein are methods of treating a human subject diagnosed with a Tauopathy, comprising administering intravenously to the human subject an amount of an antibody that specifically binds Tau, wherein the amount is a single dose to achieve a geometric mean serum AUC₍₀₋₆₇₂ₕ₎ of from about 1580 μg*hr/mL to about 122000 μg*hr/mL after administration.

Also provided herein are methods of treating a human subject diagnosed with a Tauopathy, comprising administering intravenously to the human subject an amount of an antibody that specifically binds Tau, wherein the amount is a single dose to achieve a CSF Cₘₐₓ in the range of 13.5 ng/mL to 672 ng/mL after administration to a human subject. Also provided herein are methods of treating a human subject diagnosed with a Tauopathy, comprising administering intravenously to the human subject an amount of an antibody that specifically binds Tau, wherein the amount is a single dose to achieve a geometric mean CSF Cₘₐₓ of from about 15.9 ng/mL to about 404 ng/mL after administration to a human subject.

Also provided herein are methods of treating a human subject diagnosed with a Tauopathy, comprising administering intravenously to the human subject an amount of an antibody that specifically binds Tau, wherein the amount is a single dose to achieve a CSF AUC₍₀₋₂₄ₕ₎ in the range of 159 ng*hr/mL to 7690 ng*hr/mL after administration. Also provided herein are methods of treating a human subject diagnosed with a Tauopathy, comprising administering intravenously to the human subject an amount of an antibody that specifically binds Tau, wherein the amount is a single dose to achieve a geometric mean CSF AUC₍₀₋₂₄ₕ₎ of from about 191 ng*hr/mL to about 5320 ng*hr/mL after administration.

Further provided herein are pharmaceutical compositions for treating a human subject diagnosed with a Tauopathy, comprising an antibody that specifically binds Tau, wherein the antibody that specifically binds Tau is administered to the subject as a single dose of about 3 mg/kg to about 90 mg/kg. According to some embodiments of the pharmaceutical compositions for treating a human subject diagnosed with a Tauopathy, the antibody that specifically binds Tau is administered to the subject as a single dose of about 3 mg/kg, about 10 mg/kg, about 30 mg/kg, about 60 mg/kg, or about 90 mg/kg.

According to some embodiments of the pharmaceutical compositions for treating a human subject diagnosed with a Tauopathy, the antibody that specifically binds Tau is administered to the subject at a single dose to achieve a serum Cₘₐₓ in the range of 6.29 μg/mL to 1960 μg/mL after administration. According to some embodiments of the pharmaceutical compositions for treating a human subject diagnosed with a Tauopathy, the antibody that specifically binds Tau is administered to the subject at a single dose to achieve a geometric mean serum Cₘₐₓ of from about 9.55 μg/mL to about 1450 μg/mL after administration.

According to some embodiments of the pharmaceutical compositions for treating a human subject diagnosed with a Tauopathy, the antibody that specifically binds Tau is administered to the subject at a single dose to achieve a serum AUC_{(0-inf)} in the range of 12300 μg*hr/mL to 194000 μg*hr/mL after administration. According to some embodiments of the pharmaceutical compositions for treating a human subject diagnosed with a Tauopathy, the antibody that specifically binds Tau is administered to the subject at a single dose to achieve a geometric mean serum AUC_{(0-inf)} of from about 12300 μg*hr/mL to about 130000 μg*hr/mL after administration.

According to some embodiments of the pharmaceutical compositions for treating a human subject diagnosed with a Tauopathy, the antibody that specifically binds Tau is administered to the subject at a single dose to achieve a serum AUC₍₀₋₆₇₂ₕ₎ in the range of 839 μg*hr/mL to 203000 μg*hr/mL after administration. According to some embodiments of the pharmaceutical compositions for treating a human subject diagnosed with a Tauopathy, the antibody that specifically binds Tau is administered to the subject at a single dose to achieve a geometric mean serum AUC₍₀₋₆₇₂ₕ₎ of from about 1580 μg*hr/mL to about 122000 μg*hr/mL after administration.

According to some embodiments of the pharmaceutical compositions for treating a human subject diagnosed with a Tauopathy, the antibody that specifically binds Tau is administered to the subject at a single dose to achieve a CSF Cₘₐₓ in the range of 13.5 ng/mL to 672 ng/mL after administration. According to some embodiments of the pharmaceutical compositions for treating a human subject diagnosed with a Tauopathy, the antibody that specifically binds Tau is administered to the subject at a single dose to achieve a geometric mean serum Cₘₐₓ of from about 15.9 ng/mL to about 404 ng/mL after administration.

According to some embodiments of the pharmaceutical compositions for treating a human subject diagnosed with a Tauopathy, the antibody that specifically binds Tau is administered to the subject at a single dose to achieve a CSF AUC₍₀₋₂₄ₕ₎ in the range of 159 ng*hr/mL to 7690 ng*hr/mL after administration. According to some embodiments of the pharmaceutical compositions for treating a human subject diagnosed with a Tauopathy, the antibody that specifically binds Tau is administered to the subject at a single dose to achieve a geometric mean CSF AUC₍₀₋₂₄ₕ₎ of from about 191 ng*hr/mL to about 5320 ng*hr/mL after administration.

Provided herein are intravenous dosage forms comprising an amount of antibody that specifically binds Tau, wherein the amount is a dose of about 750 mg to about 4500 mg. According to some embodiments of the intravenous dosage forms comprising an amount of an antibody that specifically binds Tau, wherein the amount is a dose of about 1500 mg to about 4500 mg, about 1500 mg to about 3000 mg, or about 3000 mg to about 4500 mg. According to some embodiments, the dose of the antibody is administered to the subject once every four weeks. According to some embodiments, intravenous dosage forms comprising an amount of an antibody that specifically binds Tau, wherein the amount is a dose of about 750 mg, about 1500 mg, about 3000 mg, or about 4500 mg, are provided.

Provided herein are intravenous dosage forms comprising an amount of an antibody that specifically binds Tau, wherein the amount is a dose to achieve a serum Cₘₐₓ in the range of 21.1 μg/mL to 655 μg/mL after administration to a human subject. Further provided herein are intravenous dosage forms comprising an amount of an antibody that specifically binds Tau, wherein the amount is a dose to achieve a geometric mean serum Cₘₐₓ of from about 35.6 μg/mL to about 509 μg/mL after administration to a human subject.

Also provided herein are intravenous dosage forms comprising an amount of an antibody that specifically binds Tau, wherein the amount is a dose to achieve a serum AUC₍₀₋₆₇₂ₕ₎ in the range of 2690 μg*hr/mL to 58900 μg*hr/mL after administration to a human subject. Further provided herein are intravenous dosage forms comprising an amount of an antibody that specifically binds Tau, wherein the amount is a dose to achieve a geometric mean serum AUC₍₀₋₆₇₂ₕ₎ of from about 5360 μg*hr/mL to about 30300 μg*hr/mL after administration to a human subject.

Also provided herein are methods of treating a human subject diagnosed with a Tauopathy, comprising administering to the human subject an amount of an antibody that specifically binds Tau, wherein the amount of the antibody that specifically binds Tau is a dose of about 750 mg to about 4500 mg. According to some embodiments of the methods, the amount of the antibody that specifically binds Tau is a dose of about 1500 mg to about 4500 mg, about 1500 mg to about 3000 mg, or about 3000 mg to about 4500 mg. According to some embodiments, the dose of the antibody is administered to the subject once every four weeks. According to some embodiments, the dose of the antibody is administered intravenously to the subject. According to some embodiments of the methods, the amount of the antibody that specifically binds Tau is a dose of about 750 mg, about 1500 mg, about 3000 mg, or about 4500 mg. According to some embodiments of the methods, the amount of the antibody that specifically binds Tau is a dose of about 750 mg, about 1500 mg, about 3000 mg, or about 4500 mg administered once every four weeks. According to some embodiments of the methods, the amount of the antibody that specifically binds Tau is a dose of about 750 mg, about 1500 mg, about 3000 mg, or about 4500 mg administered intravenously.

Provided herein are methods of treating a human subject diagnosed with a Tauopathy, comprising administering to the human subject an amount of an antibody that specifically binds Tau, wherein the amount is a dose to achieve a serum Cₘₐₓ in the range of 21.1 μg/mL to 655 μg/mL after administration to the human subject. According to some embodiments, provided herein are methods of treating a human subject diagnosed with a Tauopathy, comprising administering to the human subject an amount of an antibody that specifically binds Tau, wherein the amount is a dose to achieve a geometric mean serum Cₘₐₓ of from about 35.6 μg/mL to about 509 μg/mL after administration to the human subject.

Further provided herein are methods of treating a human subject diagnosed with a Tauopathy, comprising administering to the human subject an amount of an antibody that specifically binds Tau, wherein the amount is a dose to achieve a serum AUC₍₀₋₆₇₂ₕ₎ in the range of 2690 μg*hr/mL to 58900 μg*hr/mL after administration to the human subject. According to some embodiments are provided methods of treating a human subject diagnosed with a Tauopathy, comprising administering to the human subject an amount of an antibody that specifically binds Tau, wherein the amount is a dose to achieve a geometric mean serum AUC₍₀₋₆₇₂ₕ₎ of from about 5360 μg*hr/mL to about 30300 μg*hr/mL after administration to the human subject.

Further provided herein are pharmaceutical compositions for treating a human subject diagnosed with a Tauopathy, comprising an antibody that specifically binds Tau, wherein the antibody that specifically binds Tau is administered to the subject in an amount of about 750 to about 4500 mg. According to some embodiments of the pharmaceutical compositions, the antibody that specifically binds Tau is administered to the subject in an amount of about 1500 mg to about 4500 mg, about 1500 mg to about 3000 mg, or about 3000 mg to about 4500 mg. According to some embodiments, the antibody is administered to the subject once every four weeks. According to some embodiments, the antibody is administered intravenously to the subject. According to some embodiments of the pharmaceutical compositions, the antibody that specifically binds Tau is administered in an amount of about 750 mg, about 1500 mg, about 3000 mg, or about 4500 mg. According to some embodiments of the pharmaceutical compositions, the antibody that specifically binds Tau is administered in an amount of about 750 mg, about 1500 mg, about 3000 mg, or about 4500 mg once every four weeks. According to some embodiments of the pharmaceutical compositions, the antibody that specifically binds Tau is administered intravenously in an amount of about 750 mg, about 1500 mg, about 3000 mg, or about 4500 mg.

According to some embodiments of the pharmaceutical compositions for treating a human subject diagnosed with a Tauopathy, the antibody that specifically binds Tau is administered to the subject at a dose to achieve a serum Cₘₐₓ in the range of 21.1 μg/mL to 655 μg/mL after administration to the human subject. According to some embodiments of the pharmaceutical compositions for treating a human subject diagnosed with a Tauopathy, the antibody that specifically binds Tau is administered to the subject at a dose to achieve a geometric mean serum Cₘₐₓ of from about 35.6 μg/mL to about 509 μg/mL after administration to the human subject.

According to some embodiments of the pharmaceutical compositions for treating a human subject diagnosed with a Tauopathy, the antibody that specifically binds Tau is administered to the subject at a dose to achieve a serum AUC₍₀₋₆₇₂ₕ₎ in the range of 2690 μg*hr/mL to 58900 μg*hr/mL after administration to the human subject. According to some embodiments of the pharmaceutical compositions for treating a human subject diagnosed with a Tauopathy, the antibody that specifically binds Tau is administered to the subject at a dose to achieve a geometric mean serum AUC₍₀₋₆₇₂ₕ₎ of from about 5360 μg*hr/mL to about 30300 μg*hr/mL after administration to the human subject.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed.

In the drawings:

FIG. 1 provides a schedule of procedures and assessments for the single ascending dose (SAD) component of Study E2814-A001-001.

FIG. 2 provides an overview of the study design of the single ascending dose component of Study E2814-A001-001.

FIG. 3 provides a schedule of procedures and assessments for the multiple ascending dose (MAD) component of Study E2814-A001-001.

FIG. 4 provides an overview of the study design of the multiple ascending dose component of Study E2814-A001-001.

FIG. 5A summarizes the demographics and baseline characteristics of study subjects by cohort of the single ascending dose component of Study E2814-A001-001. FIG. 5B summarizes the demographics and baseline characteristics of study subjects by cohort of the multiple ascending dose component of Study E2814-A001-001.

FIG. 6 summarizes the geometric mean (CV %) E2814 serum PK parameters by study cohort of the single ascending dose component of Study E2814-A001-001.

FIG. 7 illustrates the mean (+SD) E2814 serum concentration-time profiles by matrix and by dose of the single ascending dose component of Study E2814-A001-001 (preliminary data).

FIG. 8 summarizes the geometric mean (CV %) E2814 serum CSF parameters by study cohort of the single ascending dose component of Study E2814-A001-001.

FIG. 9 illustrates the mean (+SD) E2814 CSF concentration-time profiles by study cohort of the single ascending dose component of Study E2814-A001-001.

FIG. 10 provides the geometric mean (gCV %) E2814 serum PK parameters following multiple IV dose administration from the multiple ascending dose component of Study E2814-A001-001.

FIG. 11A illustrates the individual CSF % bound Tau299 versus time in the single ascending dose component of Study E2814-A001-001. FIG. 11B illustrates the individual CSF % bound Tau354 versus time in the single ascending dose component of Study E2814-A001-001.

FIG. 12A illustrates the Individual CSF Bound (% of Total) MTBR-tau299 versus Time by Dose of the multiple ascending dose component of Study E2814-A001-001. FIG. 12B illustrates the Individual CSF Bound (% of Total) MTBR-tau354 versus Time by Dose of the multiple ascending dose component of Study E2814-A001-001. MAD dose cohort designations: open circles 750 mg, closed triangles 1500 mg, closed circles 3000 mg.

FIG. 13A illustrates the individual CSF bound (% of Total) MTBR-tau299 versus E2814 CSF concentration of the single ascending dose (SAD) component and multiple ascending dose (MAD) component of Study E2814-A001-001. FIG. 13B illustrates the individual CSF Bound (% of Total) MTBR-tau354 versus E2814 CSF concentration of the single ascending dose component and multiple ascending dose component of Study E2814-A001-001. SAD dose cohort designations: closed triangle 3 mg/kg, closed circle 10 mg/kg, open square 30 mg/kg, open diamond 60 mg/kg, open circle 90 mg/kg. MAD dose cohort designations: open triangles 750 mg, asterisk 1500 mg, plus sign 3000 mg. Based on preliminary data and excludes 3 PK outliers.

FIG. 14A summarizes the adverse events of the single ascending dose component of Study E2814-A001-001 by dose, severity, relatedness, and term. FIG. 14B summarizes the adverse events of the multiple ascending dose component of Study E2814-A001-001 by dose, severity, relatedness, and term.

FIG. 15 shows the mean (+SD) E2814 serum concentration-time profiles by day and faceted by dose of the multiple ascending dose component of Study E2814-A001-001.

FIG. 16 provides an overview of the study design of the Open Label, Phase 1b/2 Study (Study E2814-G000-103).

FIG. 17 provides a schedule of procedures and assessments for Study E2814-G000-103.

### DETAILED DESCRIPTION OF ILLUSTRATIVE EMBODIMENTS

The following description provides dosage forms, methods of using, and pharmaceutical compositions of antibodies that specifically bind to Tau. In some embodiments, the provided dosage forms, methods, and pharmaceutical compositions may be used to treat a Tauopathy in a subject.

The disclosed dosage forms, methods, and pharmaceutical compositions may be understood more readily by reference to the following detailed description taken in connection with the accompanying figures, which form a part of this disclosure. It is to be understood that the disclosed dosage forms, methods, and pharmaceutical compositions are not limited to those specifically described and/or shown herein, and that the terminology used herein is for the purpose of describing particular embodiments by way of example only and is not intended to be limiting of the claimed dosage forms, methods, and pharmaceutical compositions.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

The term “antibody” as used herein is meant in a broad sense and includes immunoglobulin or antibody molecules including polyclonal antibodies, monoclonal antibodies including murine, human, human-adapted, humanized, and chimeric monoclonal antibodies and antibody fragments. In general, antibodies are proteins or peptide chains that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (variable region) (VH) followed by a number of constant domains (constant regions). Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains.

Immunoglobulins can be assigned to five major classes or isotypes, depending upon the type of constant domain possessed by its heavy chain, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. The heavy chain constant domains that correspond to the different classes of immunoglobulins are called a, 6, E, y, and, respectively.

An immunoglobulin light chain variable region or heavy chain variable region consists of a “framework” region interrupted by three “antigen-binding sites”. The antigen-binding sites are defined using various terms as follows: (i) the term Complementarity Determining Regions (CDRs) is based on sequence variability (Wu and Kabat, J. Exp. Med. 132:211-250, 1970). Generally, the antigen-binding site has six CDRs; three in the VH (HCDR1, HCDR2, HCDR3), and three in the VL (LCDR1, LCDR2, LCDR3) (Kabat et al., Sequences of Proteins of Immunological Interest, 5th Ed. Public Health Service, National Institutes of Health, Bethesda, Md., 1991). The “IMGT-CDRs” as proposed by Lefranc (Lefranc et al., Dev. Comparat. Immunol. 27:55-77, 2003) are based on the comparison of V domains from immunoglobulins and T-cell receptors. The International ImMunoGeneTics (IMGT) database (www_imgt_org) provides a standardized numbering and definition of these regions. The correspondence between CDRs and IMGT delineations is described in Lefranc et al., Dev. Comparat. Immunol. 27:55-77, 2003.

Antigen-binding fragments are any proteinaceous structure that may exhibit binding affinity for a particular antigen. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation, diabodies and single-chain molecules as well as Fab, F(ab′)₂, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof” may be used to denote that a given antigen-binding fragment incorporates one or more amino acid segments of the antibody referred to in the phrase.

“Biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Specific binding” or “specifically binds” refers to the binding of an antibody to an antigen with greater affinity than for other antigens. Typically, the antibody binds to the antigen with an equilibrium dissociation constant K_{D} of about 5×10⁻⁸ M or less, for example about 5×10⁻⁹ M or less, about 1×10⁻⁹ M or less, about 1×10⁻¹⁰ M or less, or about 1×10⁻¹¹ M or less.

The term “comprising” is intended to include examples encompassed by the terms “consisting essentially of” and “consisting of”; similarly, the term “consisting essentially of” is intended to include examples encompassed by the term “consisting of.”

“Adverse event” or “AE” refers to any untoward medical occurrence in a clinical study subject administered an antibody that specifically binds Tau. An AE does not necessarily have a causal relationship with the treatment. An AE can therefore be any unfavorable and unintended sign (including an abnormal finding), symptom, or disease temporally associated with the use of a medicinal (investigational or non-investigational) product, whether or not related to the antibody that specifically binds Tau.

“Dosage” refers to the amount of the therapeutic or the drug to be taken by the subject and the frequency of the number of times the therapeutic is to be taken by the subject.

“Dose” refers to the amount or quantity of the therapeutic or the drug to be taken each time.

The term “bioequivalent” or “bioequivalence” is a term of art and is intended to be defined in accordance with Approved Drug Products with Therapeutic Equivalence Evaluations, 34th Edition, which is published by the U.S. Department of Health and Human Services, and is commonly known as the “Orange Book.” Bioequivalence of different formulation of the same drug substance involves equivalence with respect to the rate and extent of drug absorption. The extent and rate of absorption of the test formulation is compared to a reference formulation in order to determine whether the two formulations are bioequivalent. The standard bioequivalence study is conducted in crossover fashion by extensive testing which includes administering single doses of the test and reference drugs to a number of volunteers, usually 12 to 24 healthy normal adults, and then measuring the blood, serum, or plasma levels of the drug over time. Detailed guidelines for establishing the bioequivalence of a formulation with a reference formulation have been published by the FDA Office of Generic Drugs, Division of Bioequivalence.

Two dosage forms whose rate and extent of absorption differ by −20%/+25% or less are generally considered “bioequivalent.” Another approach for average bioequivalence involves the calculation of a 90% confidence interval for the ratio of the averages (population geometric means) of the measures for the test and reference products. To establish BE, the calculated confidence interval should fall within usually 80-125% for the ratio of the product averages. In addition to this general approach, the others approach, including (1) logarithmic transformation of pharmacokinetic data, (2) methods to evaluate sequence effects and (3) methods to evaluate outlier data, may be useful for the establishment of bioequivalence. For example, in the above (1) the confidence interval should fall within usually 80-125% for the difference in the mean value of the logarithmic converted PK parameter.

“mg/kg” refers to dosing of a drug in milligrams per kilogram of subject body mass.

The term “a mean” refers to a geometric mean. The pharmacokinetic parameters such as “a mean Cₘₐₓ” or “a mean AUC” refer to the geometric mean value of a Cₘₐₓ or an AUC.

The term “treating” or “treatment” refers to any success or indicia of success in the attenuation or amelioration of an injury, pathology or condition, including any objective or subjective parameter such as abatement, remission, diminishing of a symptom or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations. In a particular embodiment, the symptom of a Tauopathy is an impairment in cognition. In a specific embodiment, the symptom of a Tauopathy is an impairment in learning and/or memory. In a specific embodiment, the symptom of a Tauopathy is a long-term memory loss. In a specific embodiment, the symptom of a Tauopathy is dementia. In some embodiments, the symptom of a Tauopathy is confusion, irritability, aggression, mood swings, or a language impairment. In some embodiments, the symptom of a Tauopathy is an impairment or loss of one or more cognitive functions such as reasoning, situational judgment, memory capacity, and/or learning.

“Treatment regimen” refers to a combination of dosage, frequency of administration, and/or duration of treatment. “Effective treatment regimen” refers to a treatment regimen that will offer beneficial response to a patient receiving the treatment. An “effective amount” or “effective dose” of an agent refers to an amount or dose effective, for periods of time necessary, to achieve the desired result. For example, a “therapeutically effective amount” refers to an amount of antibody effective, for the period of time necessary, to produce a therapeutic effect in a human subject.

As used herein, “therapeutic effect” is a consequence of a medical treatment of any kind, the results of which are judged to be desirable and beneficial. This is true whether the result was expected, unexpected, or even an unintended consequence of the treatment. A therapeutic effect may also be an objectively identifiable improvement as noted by the clinician or other qualified observer. In a particular embodiment, the therapeutic effect of an antibody that specifically binds Tau can be detected by evaluating the binding the antibody to MTBR-Tau. MTBR-Tau fragments are measurable in cerebrospinal fluid (CSF) from patients with AD (Alzheimer's & Dementia Volume 15, Issue 7, Supplement, July 2019, Pages P1598-P1599). MTBR-Tau is significantly increased in CSF of patients with AD compared to healthy adults.

As used herein, “administering” and similar terms indicate a procedure by which a pharmaceutical formulation is injected into a subject.

The term “subject” as used herein is intended to mean any animal, in particular, mammals. The methods are applicable to human and nonhuman animals, although most preferably with humans. In some embodiments, the subject has a mutation in at least one of three genes, Amyloid precursor protein (APP), Presenilin 1 (PSEN1), or Presenilin 2 (PSEN2). In some embodiments, the subject has a mutation in the APP gene. In some embodiments, the subject has a mutation in the PSEN1 gene. In some embodiments, the subject has a mutation in the PSEN2 gene. Specific mutations in the APP, PSEN1, or PSEN2 genes that contribute to DIAD are known in the art (e.g., Cruts & Van Broeckhoven, Hum Mutat. 1998; 11(3):183-90; Cruts, Theuns, & Van Broeckhoven, Hum Mutat., 2012 September; 33(9):1340-4; Ryman et al., Symptom onset in autosomal dominant Alzheimer disease: a systematic review and metaanalysis. Neurology, 83(3), 253-260; Sherva, R., & Kowall, N. (2018). Genetics of Alzheimer disease—UpToDate. In J. Wilterdink (Ed.), UpToDate. Retrieved from www_uptodate_com/contents/genetics-of-alzheimerdisease?sectionName=GENETIC%20TESTING&topicRef=5071&anchor=H900056&source=see_link#H900056). “Subject” and “patient” can be used interchangeably herein.

Provided herein are intravenous dosage forms comprising an amount of antibody that specifically binds Tau, wherein the amount is a single dose of about 3 mg/kg to about 90 mg/kg. According to some embodiments, intravenous dosage forms comprising an amount of an antibody that specifically binds Tau, wherein the amount is a single dose of about 3 mg/kg, about 10 mg/kg, about 30 mg/kg, about 60 mg/kg or about 90 mg/kg, are provided.

Provided herein are intravenous dosage forms comprising an amount of an antibody that specifically binds Tau, wherein the amount is a single dose to achieve a serum Cₘₐₓ in the range of 6.29 μg/mL to 1960 μg/mL after administration to a human subject. Further provided herein are intravenous dosage forms comprising an amount of an antibody that specifically binds Tau, wherein the amount is a single dose to achieve a geometric mean serum Cₘₐₓ of from about 9.55 μg/mL to about 1450 μg/mL after administration to a human subject.

Further provided herein are intravenous dosage forms comprising an amount of an antibody that specifically binds Tau, wherein the amount is a single dose to achieve a serum AUC_{(0-inf)} in the range of 12300 μg*hr/mL to 194000 μg*hr/mL after administration to a human subject. Further provided herein are intravenous dosage forms comprising an amount of an antibody that specifically binds Tau, wherein the amount is a single dose to achieve a geometric mean serum AUC_{(0-inf)} of from about 12300 μg*hr/mL to about 130000 μg*hr/mL after administration to a human subject.

Further provided herein are intravenous dosage forms comprising an amount of an antibody that specifically binds Tau, wherein the amount is a single dose to achieve a serum AUC₍₀₋₆₇₂ₕ₎ in the range of 839 μg*hr/mL to 203000 μg*hr/mL after administration to a human subject. Further provided herein are intravenous dosage forms comprising an amount of an antibody that specifically binds Tau, wherein the amount is a single dose to achieve a geometric mean serum AUC₍₀₋₆₇₂ₕ₎ of from about 1580 μg*hr/mL to about 122000 μg*hr/mL after administration to a human subject.

Provided herein are intravenous dosage forms comprising an amount of an antibody that specifically binds Tau, wherein the amount is a single dose to achieve a CSF Cₘₐₓ in the range of 13.5 ng/mL to 672 ng/mL after administration to a human subject. Further provided herein are intravenous dosage forms comprising an amount of an antibody that specifically binds Tau, wherein the amount is a single dose to achieve a geometric mean CSF Cₘₐₓ of from about 15.9 ng/mL to about 404 ng/mL after administration to a human subject.

Further provided herein are intravenous dosage forms comprising an amount of an antibody that specifically binds Tau, wherein the amount is a single dose to achieve a CSF AUC₍₀₋₂₄ₕ₎ in the range of 159 ng*hr/mL to 7690 ng*hr/mL after administration to a human subject. Further provided herein are intravenous dosage forms comprising an amount of an antibody that specifically binds Tau, wherein the amount is a single dose to achieve a geometric mean CSF AUC₍₀₋₂₄ₕ₎ of from about 191 ng*hr/mL to about 5320 ng*hr/mL after administration to a human subject.

Provided herein are intravenous dosage forms comprising an amount of antibody that specifically binds Tau, wherein the amount is a dose of about 750 mg to about 4500 mg. According to some embodiments of the intravenous dosage forms comprising an amount of an antibody that specifically binds Tau, wherein the amount is a dose of about 1500 mg to about 4500 mg, about 1500 mg to about 3000 mg, or about 3000 mg to about 4500 mg. According to some embodiments, the dose of the antibody is administered to the subject once every four weeks. According to some embodiments, intravenous dosage forms comprising an amount of an antibody that specifically binds Tau, wherein the amount is a dose of about 750 mg, about 1500 mg, about 3000 mg, or about 4500 mg, are provided.

Provided herein are intravenous dosage forms comprising an amount of an antibody that specifically binds Tau, wherein the amount is a dose to achieve a serum Cₘₐₓ in the range of 21.1 μg/mL to 655 μg/mL after administration to a human subject. Further provided herein are intravenous dosage forms comprising an amount of an antibody that specifically binds Tau, wherein the amount is a dose to achieve a geometric mean serum Cₘₐₓ of from about 35.6 μg/mL to about 509 μg/mL after administration to a human subject.

Also provided herein are intravenous dosage forms comprising an amount of an antibody that specifically binds Tau, wherein the amount is a dose to achieve a serum AUC₍₀₋₆₇₂ₕ₎ in the range of 2690 μg*hr/mL to 58900 μg*hr/mL after administration to a human subject. Further provided herein are intravenous dosage forms comprising an amount of an antibody that specifically binds Tau, wherein the amount is a dose to achieve a geometric mean serum AUC₍₀₋₆₇₂ₕ₎ of from about 5360 μg*hr/mL to about 30300 μg*hr/mL after administration to a human subject.

Also provided herein are methods of treating a human subject diagnosed with a Tauopathy, comprising administering intravenously to the human subject an amount of an antibody that specifically binds Tau, wherein the amount is a single dose of about 3 mg/kg to about 90 mg/kg. According to some embodiments of the methods, the amount of the antibody that specifically binds Tau is a single dose of about 3 mg/kg, about 10 mg/kg, about 30 mg/kg, about 60 mg/kg, or about 90 mg/kg.

According to some embodiments of the methods of treating a human subject diagnosed with a Tauopathy, the amount of the antibody that specifically binds Tau is a single dose to achieve a serum Cₘₐₓ in the range of 6.29 μg/mL to 1960 μg/mL after administration. According to some embodiments of the methods of treating human subject diagnosed with a Tauopathy, the amount of the antibody that specifically binds Tau is a single dose to achieve a geometric mean serum Cₘₐₓ of from about 9.55 μg/mL to about 1450 μg/mL after administration.

According to some embodiments of the methods of treating a human subject diagnosed with a Tauopathy, the amount of the antibody that specifically binds Tau is a single dose to achieve a serum AUC_{(0-inf)} in the range of 12300 μg*hr/mL to 194000 μg*hr/mL after administration. According to some embodiments of the methods of treating a human subject diagnosed with a Tauopathy, the amount of the antibody that specifically binds Tau is a single dose to achieve a geometric mean serum AUC_{(0-inf)} of from about 12300 μg*hr/mL to about 130000 μg*hr/mL after administration.

According to some embodiments of the methods of treating a human subject diagnosed with a Tauopathy, the amount of the antibody that specifically binds Tau is a single dose to achieve a serum AUC₍₀₋₆₇₂ₕ₎ in the range of 839 μg*hr/mL to 203000 μg*hr/mL after administration. According to some embodiments of the methods of treating a human subject diagnosed with a Tauopathy, the amount of the antibody that specifically binds Tau is a single dose to achieve a geometric mean serum AUC₍₀₋₆₇₂ₕ₎ of from about 1580 μg*hr/mL to about 122000 μg*hr/mL after administration.

According to some embodiments of the methods of treating a human subject diagnosed with a Tauopathy, the amount of the antibody that specifically binds Tau is a single dose to achieve a CSF Cₘₐₓ in the range of 13.5 ng/mL to 672 ng/mL after administration to a human subject. According to some embodiments of the methods of treating a human subject diagnosed with a Tauopathy, the amount of the antibody that specifically binds Tau is a single dose to achieve a geometric mean CSF Cₘₐₓ of from about 15.9 ng/mL to about 404 ng/mL after administration to a human subject.

According to some embodiments of the methods of treating a human subject diagnosed with a Tauopathy, the amount of the antibody that specifically binds Tau is a single dose to achieve a CSF AUC₍₀₋₂₄ₕ₎ in the range of 159 ng*hr/mL to 7690 ng*hr/mL after administration. According to some embodiments of the methods of treating a human subject diagnosed with a Tauopathy, the amount of the antibody that specifically binds Tau is a single dose to achieve a geometric mean CSF AUC₍₀₋₂₄ₕ₎ of from about 191 ng*hr/mL to about 5320 ng*hr/mL after administration.

Also provided herein are methods of treating a human subject diagnosed with a Tauopathy, comprising administering to the human subject an amount of an antibody that specifically binds Tau, wherein the amount of the antibody that specifically binds Tau is a dose of about 750 mg to about 4500 mg. According to some embodiments of the methods, the amount of the antibody that specifically binds Tau is a dose of about 1500 mg to about 4500 mg, about 1500 mg to about 3000 mg, or about 3000 mg to about 4500 mg. According to some embodiments, the dose of the antibody is administered to the subject once every four weeks. According to some embodiments, the dose of the antibody is intravenously administered to the subject. According to some embodiments of the methods, the amount of the antibody that specifically binds Tau is a dose of about 750 mg, about 1500 mg, about 3000 mg, or about 4500 mg. According to some embodiments of the methods, the amount of the antibody that specifically binds Tau is a dose of about 750 mg, about 1500 mg, about 3000 mg, or about 4500 mg administered once every four weeks. According to some embodiments of the methods, the amount of the antibody that specifically binds Tau is a dose of about 750 mg, about 1500 mg, about 3000 mg, or about 4500 mg administered intravenously.

According to some embodiments of the methods, the amount of the antibody that specifically binds Tau is a dose of about 750 mg administered once every four weeks for three administration cycles or twelve weeks followed by about 1500 mg administered once every four weeks. According to some embodiments of the methods, the amount of the antibody that specifically binds Tau is a dose of about 1500 mg administered once every four weeks for three administration cycles or twelve weeks followed by about 3000 mg administered once every four weeks. According to some embodiments of the methods, the amount of the antibody that specifically binds Tau is a dose of about 3000 mg administered once every four weeks for three administration cycles or twelve weeks followed by about 4500 mg administered once every four weeks. According to some embodiments of the methods, the amount of the antibody that specifically binds Tau is a dose of about 750 mg administered once every four weeks for three administration cycles or twelve weeks followed by 1500 mg administered once every four weeks for three administration cycles or twelve weeks followed by about 3000 mg administered once every four weeks. According to some embodiments of the methods, the amount of the antibody that specifically binds Tau is a dose of about 750 mg administered once every four weeks for three administration cycles or twelve weeks followed by 1500 mg administered once every four weeks for three administration cycles or twelve weeks followed by about 3000 mg administered once every four weeks for three administration cycles or twelve weeks followed by about 4500 mg every four weeks.

According to some embodiments of the methods of treating a human subject diagnosed with a Tauopathy, the amount of the antibody that specifically binds Tau is a dose to achieve a serum Cₘₐₓ in the range of 21.1 μg/mL to 655 μg/mL after administration to the human subject. According to some embodiments of the methods of treating a human subject diagnosed with a Tauopathy, the amount of the antibody that specifically binds Tau is a dose to achieve a geometric mean serum Cₘₐₓ of from about 35.6 μg/mL to about 509 μg/mL after administration to the human subject.

According to some embodiments of the methods of treating a human subject diagnosed with a Tauopathy, the amount is a dose to achieve a serum AUC₍₀₋₆₇₂ₕ₎ in the range of 2690 μg*hr/mL to 58900 μg*hr/mL after administration to the human subject. According to some embodiments of the methods of treating a human subject diagnosed with a Tauopathy, the amount of the antibody that specifically binds Tau is a dose to achieve a geometric mean serum AUC₍₀₋₆₇₂ₕ₎ of from about 5360 μg*hr/mL to about 30300 μg*hr/mL after administration to the human subject.

Further provided herein are pharmaceutical compositions for treating a human subject diagnosed with a Tauopathy, comprising an antibody that specifically binds Tau, wherein the antibody that specifically binds Tau is administered to the subject as a single dose of about 3 mg/kg to about 90 mg/kg. According to some embodiments of the pharmaceutical compositions for treating a human subject diagnosed with a Tauopathy, the antibody that specifically binds Tau is administered to the subject as a single dose of about 3 mg/kg, about 10 mg/kg, about 30 mg/kg, about 60 mg/kg, or about 90 mg/kg.

According to some embodiments of the pharmaceutical compositions for treating a human subject diagnosed with a Tauopathy, the antibody that specifically binds Tau is administered to the subject at a single dose to achieve a serum Cₘₐₓ in the range of 6.29 μg/mL to 1960 μg/mL after administration. According to some embodiments of the pharmaceutical compositions for treating a human subject diagnosed with a Tauopathy, the antibody that specifically binds Tau is administered to the subject at a single dose to achieve a geometric mean serum Cₘₐₓ of from about 9.55 μg/mL to about 1450 μg/mL after administration.

According to some embodiments of the pharmaceutical compositions for treating a human subject diagnosed with a Tauopathy, the antibody that specifically binds Tau is administered to the subject at a single dose to achieve a serum AUC_{(0-inf)} in the range of 12300 μg*hr/mL to 194000 μg*hr/mL after administration. According to some embodiments of the pharmaceutical compositions for treating a human subject diagnosed with a Tauopathy, the antibody that specifically binds Tau is administered to the subject at a single dose to achieve a geometric mean serum AUC_{(0-inf)} of from about 12300 μg*hr/mL to about 130000 μg*hr/mL after administration.

According to some embodiments of the pharmaceutical compositions for treating a human subject diagnosed with a Tauopathy, the antibody that specifically binds Tau is administered to the subject at a single dose to achieve a serum AUC₍₀₋₆₇₂ₕ₎ in the range of 839 μg*hr/mL to 203000 μg*hr/mL after administration. According to some embodiments of the pharmaceutical compositions for treating a human subject diagnosed with a Tauopathy, the antibody that specifically binds Tau is administered to the subject at a single dose to achieve a geometric mean serum AUC₍₀₋₆₇₂ₕ₎ of from about 1580 μg*hr/mL to about 122000 μg*hr/mL after administration.

According to some embodiments of the pharmaceutical compositions for treating a human subject diagnosed with a Tauopathy, the antibody that specifically binds Tau is administered to the subject at a single dose to achieve a CSF Cₘₐₓ in the range of 13.5 ng/mL to 672 ng/mL after administration. According to some embodiments of the pharmaceutical compositions for treating a human subject diagnosed with a Tauopathy, the antibody that specifically binds Tau is administered to the subject at a single dose to achieve a geometric mean serum Cₘₐₓ of from about 15.9 ng/mL to about 404 ng/mL after administration.

According to some embodiments of the pharmaceutical compositions for treating a human subject diagnosed with a Tauopathy, the antibody that specifically binds Tau is administered to the subject at a single dose to achieve a CSF AUC₍₀₋₂₄ₕ₎ in the range of 159 ng*hr/mL to 7690 ng*hr/mL after administration. According to some embodiments of the pharmaceutical compositions for treating a human subject diagnosed with a Tauopathy, the antibody that specifically binds Tau is administered to the subject at a single dose to achieve a geometric mean CSF AUC₍₀₋₂₄ₕ₎ of from about 191 ng*hr/mL to about 5320 ng*hr/mL after administration.

Further provided herein are pharmaceutical compositions for treating a human subject diagnosed with a Tauopathy, comprising an antibody that specifically binds Tau, wherein the antibody that specifically binds Tau is administered to the subject in an amount of about 750 to about 4500 mg. According to some embodiments of the pharmaceutical compositions, the antibody that specifically binds Tau is administered to the subject in an amount of about 1500 mg to about 4500 mg, about 1500 mg to about 3000 mg, or about 3000 mg to about 4500 mg. According to some embodiments, the antibody is administered to the subject once every four weeks. According to some embodiments, the antibody is administered intravenously to the subject. According to some embodiments of the pharmaceutical compositions, the antibody that specifically binds Tau is administered in an amount of about 750 mg, about 1500 mg, about 3000 mg, or about 4500 mg. According to some embodiments of the pharmaceutical compositions, the antibody that specifically binds Tau is administered in an amount of about 750 mg, about 1500 mg, about 3000 mg, or about 4500 mg once every four weeks. According to some embodiments of the pharmaceutical compositions, the antibody that specifically binds Tau is administered intravenously in an amount of about 750 mg, about 1500 mg, about 3000 mg, or about 4500 mg. According to some embodiments of the pharmaceutical compositions, the antibody that specifically binds Tau is administered in an amount of about 750 mg once every four weeks for three administration cycles or twelve weeks followed by about 1500 mg administered once every four weeks. According to some embodiments of the pharmaceutical compositions, the antibody that specifically binds Tau is administered in an amount of about 1500 mg once every four weeks for three administration cycles or twelve weeks followed by about 3000 mg administered once every four weeks. According to some embodiments of the pharmaceutical compositions, the antibody that specifically binds Tau is administered in an amount of about 3000 mg administered once every four weeks for three administration cycles or twelve weeks followed by about 4500 mg administered once every four weeks. According to some embodiments of the pharmaceutical compositions, the antibody that specifically binds Tau is administered in an amount of about 750 mg once every four weeks for three administration cycles or twelve weeks followed by 1500 mg administered once every four weeks for three administration cycles or twelve weeks followed by about 3000 mg administered once every four weeks. According to some embodiments of the pharmaceutical compositions, the antibody that specifically binds Tau is administered in an amount of about 750 mg administered once every four weeks for three administration cycles or twelve weeks followed by 1500 mg administered once every four weeks for three administration cycles or twelve weeks followed by about 3000 mg administered once every four weeks for three administration cycles or twelve weeks followed by about 4500 mg every four weeks.

According to some embodiments of the pharmaceutical compositions for treating a human subject diagnosed with a Tauopathy, the antibody that specifically binds Tau is administered to the subject at a dose to achieve a serum Cₘₐₓ in the range of 21.1 μg/mL to 655 μg/mL after administration to the human subject. According to some embodiments of the pharmaceutical compositions for treating a human subject diagnosed with a Tauopathy, the antibody that specifically binds Tau is administered to the subject at a dose to achieve a geometric mean serum Cₘₐₓ of from about 35.6 μg/mL to about 509 μg/mL after administration to the human subject.

According to some embodiments of the pharmaceutical compositions for treating a human subject diagnosed with a Tauopathy, the antibody that specifically binds Tau is administered to the subject at a dose to achieve a serum AUC₍₀₋₆₇₂ₕ₎ in the range of 2690 μg*hr/mL to 58900 μg*hr/mL after administration to the human subject. According to some embodiments of the pharmaceutical compositions for treating a human subject diagnosed with a Tauopathy, the antibody that specifically binds Tau is administered to the subject at a dose to achieve a geometric mean serum AUC₍₀₋₆₇₂ₕ₎ of from about 5360 μg*hr/mL to about 30300 μg*hr/mL after administration to the human subject.

According to some embodiments, the provided dosage forms, methods, and pharmaceutical compositions may be used to treat a Tauopathy in a subject. Exemplary Tauopathies that can be treated with the disclosed anti-Tau antibodies include Alzheimer's disease (AD), progressive supranuclear palsy (PSP), and frontotemporal dementia (FTD). An exemplary FTD that can be treated is Pick's disease (PiD). An exemplary AD that can be treated is Dominantly Inherited AD (DIAD) or sporadic AD.

According to certain embodiments of the provided dosage forms, methods, and pharmaceutical compositions, the antibody that specifically binds Tau (also referred to herein as an “anti-Tau antibody”) comprises a heavy chain variable domain (VH), light chain variable domain (VL), and/or complementarity determining regions (CDRs) comprising the amino acid sequences as set forth in Tables 1 to 5. In certain exemplary embodiments, the anti-Tau antibody that can be used in the context of the disclosed dosage forms, methods, and pharmaceutical compositions comprises the heavy chain complementarity determining regions (HCDRs) of a heavy chain variable domain (VH) comprising the amino acid sequence of SEQ ID NO: 2 and the light chain complementarity determining regions (LCDRs) of a light chain variable region (VL) comprising the amino acid sequence of SEQ ID NO: 5. According to certain embodiments, the anti-Tau antibody comprises three HCDRs (HCDR1, HCDR2 and HCDR3) and three LCDRs (LCDR1, LCDR2 and LCDR3), wherein the HCDR1 comprises the amino acid sequence of SEQ ID NO: 7; the HCDR2 comprises the amino acid sequence of SEQ ID NO: 8; the HCDR3 comprises the amino acid sequence of SEQ ID NO: 9; the LCDR1 comprises the amino acid sequence of SEQ ID NO: 10; the LCDR2 comprises the amino acid sequence of SEQ ID NO: 11; and the LCDR3 comprises the amino acid sequence of SEQ ID NO: 12, wherein the CDRs are defined according to the method of Kabat. According to certain embodiments, the anti-Tau antibody comprises three HCDRs (HCDR1, HCDR2 and HCDR3) and three LCDRs (LCDR1, LCDR2 and LCDR3), wherein the HCDR1 comprises the amino acid sequence of SEQ ID NO: 13; the HCDR2 comprises the amino acid sequence of SEQ ID NO: 14; the HCDR3 comprises the amino acid sequence of SEQ ID NO: 15; the LCDR1 comprises the amino acid sequence of SEQ ID NO: 16; the LCDR2 comprises the amino acid sequence of SEQ ID NO: 17; and the LCDR3 comprises the amino acid sequence of SEQ ID NO: 18, wherein the CDRs are defined according to the IMGT method. In yet other embodiments, the anti-Tau antibody comprises a VH comprising SEQ ID NO: 2 and an VL comprising SEQ ID NO: 5. In certain embodiments, the anti-Tau antibody comprises a heavy chain comprising the amino acid sequence of SEQ ID NO: 1 and/or a light chain comprising the amino acid sequence of SEQ ID NO: 4. An exemplary anti-Tau antibody comprising a heavy chain comprising the amino acid sequence of SEQ ID NO: 1 and a light chain comprising the amino acid sequence of SEQ ID NO: 4 is antibody E2814, also known as antibody 7G6-HCzu25-LCzu18 disclosed in Intl. Publ. No. WO2019/077500, incorporated herein by reference in its entirety. According to certain exemplary embodiments, the anti-Tau antibody is antibody E2814 or a biosimilar thereof.

TABLE 1
E2814 heavy chain and light chain sequences

| | E2814 Heavy Chain | E2814 Heavy Chain |
|---|---|---|
| E2814 Heavy Chain | Variable Domain | Constant Domain |
| Amino Acid Sequence | amino acid sequence | amino acid sequence |
| EVQLLESGGGLVQPGGSLRLSCAASGY | EVQLLESGGGLVQPGGSLR | ASTKGPSVFPLAPSSKSTSGGT |
| TFTTYWITWVRQAPGKGLEWVSDIYPG | LSCAASGYTFTTYWITWVR | AALGCLVKDYFPEPVTVSWNSG |
| SSISNYNEKFKSRFTISVDNSKNTLYL | QAPGKGLEWVSDIYPGSSI | ALTSGVHTFPAVLQSSGLYSLS |
| QMNSLRAEDTAVYYCAREDGYDAWFAY | SNYNEKFKSRFTISVDNSK | SVVTVPSSSLGTQTYICNVNHK |
| WGQGTLVTVSSASTKGPSVFPLAPSSK | NTLYLQMNSLRAEDTAVYY | PSNTKVDKKVEPKSCDKTHTCP |
| STSGGTAALGCLVKDYFPEPVTVSWNS | CAREDGYDAWFAYWGQGTL | PCPAPELLGGPSVFLFPPKPKD |
| GALTSGVHTFPAVLQSSGLYSLSSVVT | VTVSS | TLMISRTPEVTCVVVDVSHEDP |
| VPSSSLGTQTYICNVNHKPSNTKVDKK | [SEQ ID NO: 2] | EVKFNWYVDGVEVHNAKTKPRE |
| VEPKSCDKTHTCPPCPAPELLGGPSVQ | | EQYNSTYRVVSVLTVLHQDWLN |
| LFPPKPKDTLMISRTPEVTCVVVDVSH | | GKEYKCKVSNKALPAPIEKTIS |
| EDPEVKFNWYVDGVEVHNAKTKPREEQ | | KAKGQPREPQVYTLPPSRDELT |
| YNSTYRVVSVLTVLHQDWLNGKEYKCK | | KNQVSLTCLVKGFYPSDIAVEW |
| VSNKALPAPIEKTISKAKGQPREPQVY | | ESNGQPENNYKTTPPVLDSDGS |
| TLPPSRDELTKNQVSLTCLVKGFYPSD | | FFLYSKLTVDKSRWQQGNVFSC |
| IAVEWESNGQPENNYKTTPPVLDSDGS | | SVMHEALHNHYTQKSLSLSPGK |
| FFLYSKLTVDKSRWQQGNVFSCSVMHE | | [SEQ ID NO: 3] |
| ALHNHYTQKSLSLSPGK [SEQ ID | | |
| NO: 1] | | |
| DIQMTQSPSSLSASVGDRVTITCRSSQ | DIQMTQSPSSLSASVGDRV | RTVAAPSVFIFPPSDEQLKSGT |
| SILHSNGNTYLEWYQQKPGKAPKLLIS | TITCRSSQSILHSNGNTYL | ASVVCLLNNFYPREAKVQWKVD |
| KVSNRFSGVPSRFSGSGSGTDFTLTIS | EWYQQKPGKAPKLLISKVS | NALQSGNSQESVTEQDSKDSTY |
| SLQPEDFATYYCFQGSHVPFTFGQGTK | NRFSGVPSRFSGSGSGTDF | SLSSTLTLSKADYEKHKVYACE |
| LEIKRTVAAPSVFIFPPSDEQLKSGTA | TLTISSLQPEDFATYYCFQ | VTHQGLSSPVTKSFNRGEC |
| SVVCLLNNFYPREAKVQWKVDNALQSG | GSHVPFTFGQGTKLEIK | [SEQ ID NO: 6] |
| NSQESVTEQDSKDSTYSLSSTLTLSKA | [SEQ ID NO: 5] | |
| DYEKHKVYACEVTHQGLSSPVTKSENR | | |
| GEC [SEQ ID NO: 4] | | |

TABLE 2
E2814 VH numbered according to Kabat

| VH CDR1 | VH CDR2 | VH CDR3 |
|---|---|---|
| Amino Acid Sequence | Amino Acid Sequence | Amino Acid Sequence |
| TYWIT | DIYPGSSISNYNEKFKS | EDGYDAWFAY |
| [SEQ ID NO: 7] | [SEQ ID NO: 8] | [SEQ ID NO: 9] |

TABLE 3
E2814 VL numbered according to Kabat

| VL CDR1 | VL CDR2 | VL CDR3 |
|---|---|---|
| Amino Acid Sequence | Amino Acid Sequence | Amino Acid Sequence |
| RSSQSILHSNGNTYLE | KVSNRFS | FQGSHVPFT |
| [SEQ ID NO: 10] | [SEQ ID NO: 11] | [SEQ ID NO: 12] |

TABLE 4
E2814 VH numbered according to IMGT

| VH CDR1 | VH CDR2 | VH CDR3 |
|---|---|---|
| Amino Acid Sequence | Amino Acid Sequence | Amino Acid Sequence |
| GYTFTTYW | IYPGSSIS | AREDGYDAWFAY |
| [SEQ ID NO: 13] | [SEQ ID NO: 14] | [SEQ ID NO: 15] |

TABLE 5
E2814 VL numbered according to IMGT

| VL CDR1 | VL CDR2 | VL CDR3 |
|---|---|---|
| Amino Acid Sequence | Amino Acid Sequence | Amino Acid Sequence |
| QSILHSNGNTY | KVS | FQGSHVPFT |
| [SEQ ID NO: 16] | [SEQ ID NO: 17] | [SEQ ID NO: 18] |

In a further aspect, the invention provides pharmaceutical formulations comprising any of the antibodies that specifically bind Tau as described herein, e.g., for use in any of the methods provided herein. In some embodiments, a pharmaceutical formulation comprises any of the antibodies that specifically bind Tau provided herein and a pharmaceutically acceptable carrier, diluent, and/or excipient (Remington's Pharmaceutical Sciences 16th edition, Osol, A. Ed. (1980)). Pharmaceutically acceptable carriers, diluents, and excipients are generally nontoxic to recipients at the dosages and concentrations employed. The formulations to be used for in vivo administration are generally sterile. Any of the antibodies that specifically bind Tau (or formulations thereof) provided herein may be used in the disclosed methods.

In certain embodiments, an anti-Tau antibody for use in a method of treatment of a Tauopathy is provided.

According to some embodiments, the dose of anti-Tau antibody for use in a method of treatment of a Tauopathy is about 3 mg/kg to about 90 mg/kg. According to some embodiments, the dose of anti-Tau antibody for use in a method of treatment of a Tauopathy is about 3 mg/kg to about 10 mg/kg, about 10 mg/kg to about 30 mg/kg, about 30 mg/kg to about 60 mg/kg, or about 60 mg/kg to about 90 mg/kg. For example, the dose of the anti-Tau antibody may be about 3 mg/kg, about 10 mg/kg, about 30 mg/kg, about 60 mg/kg, or about 90 mg/kg. In certain embodiments, the Tauopathy is any one of the Tauopathies described above.

In a further aspect, also provided herein is the use of an anti-Tau antibody as described herein in the manufacture or preparation of a medicament. In some embodiments, the medicament is for treatment of a Tauopathy. According to some embodiments, the medicament comprises a dose of anti-Tau antibody of about 3 mg/kg to about 90 mg/kg. According to some embodiments, the medicament comprises a dose of anti-Tau antibody of about 3 mg/kg to about 10 mg/kg, about 10 mg/kg to about 30 mg/kg, about 30 mg/kg to about 60 mg/kg, or about 60 mg/kg to about 90 mg/kg. For example, the dose of the anti-Tau antibody may be about 3 mg/kg, about 10 mg/kg, about 30 mg/kg, about 60 mg/kg, or about 90 mg/kg. In certain embodiments, the Tauopathy is any one of the Tauopathies described above.

According to some embodiments, the dose of anti-Tau antibody for use in a method of treatment of a Tauopathy is about 750 mg to about 4500 mg. According to some embodiments, the dose of anti-Tau antibody for use in a method of treatment of a Tauopathy is about 1500 mg to about 4500 mg, about 1500 mg to about 3000 mg, or about 3000 mg to about 4500 mg. For example, the dose of the anti-Tau antibody may be about 750 mg, about 1500 mg, about 3000 mg, or about 4500 mg. In certain embodiments, the Tauopathy is any one of the Tauopathies described above.

In a further aspect, also provided herein is the use of an anti-Tau antibody as described herein in the manufacture or preparation of a medicament. In some embodiments, the medicament is for treatment of a Tauopathy. According to some embodiments, the medicament comprises a dose of anti-Tau antibody of about 750 mg to about 4500 mg. According to some embodiments, the medicament comprises a dose of anti-Tau antibody of about 1500 mg to about 4500 mg, about 1500 mg to about 3000 mg, or about 3000 mg to about 4500 mg. For example, the dose of the anti-Tau antibody may be about 750 mg, about 1500 mg, about 3000 mg, or about 4500 mg. In certain embodiments, the Tauopathy is any one of the Tauopathies described above.

Suitable routes of administration include parenteral administration. According to some aspects of the invention, an anti-Tau antibody as described herein is administered parenterally, e.g. by injections, such as intravenous injection.

In some aspects, the anti-Tau antibody as described herein is administered in a single administration.

Depending on the type and severity of the disease, about 3 mg/kg to about 90 mg/kg of the anti-Tau antibody can be administered to the patient in a single administration. Exemplary doses of the anti-Tau antibody to be administered per treatment session or visit are about 3 mg/kg, about 10 mg/kg, about 30 mg/kg, about 60 mg/kg, or about 90 mg/kg. According to some embodiments, the anti-Tau antibody is administered to the subject as a dose of about 3 mg/kg to about 10 mg/kg, about 10 mg/kg to about 30 mg/kg, about 30 mg/kg to about 60 mg/kg, or about 60 mg/kg to about 90 mg/kg.

Depending on the type and severity of the disease, about 750 mg to about 4500 mg of the anti-Tau antibody can be administered to the patient in a single administration. Exemplary doses of the anti-Tau antibody to be administered per treatment session or visit are about 750 mg, about 1500 mg, about 3000 mg, or about 4500 mg. According to some embodiments, the anti-Tau antibody is administered to the subject once every four weeks. According to some embodiments, the anti-Tau antibody is administered to the subject as a dose of about 1500 mg to about 4500 mg, about 1500 mg to about 3000 mg, or about 3000 mg to about 4500 mg. According to some embodiments, the anti-Tau antibody is administered to the subject as a dose of about 750 mg, about 1500 mg, about 3000 mg, or about 4500 mg. According to some embodiments, the anti-Tau antibody is administered to the subject as a dose of about 750 mg, about 1500 mg, about 3000 mg, or about 4500 mg once every four weeks.

According to some embodiments, the dose of anti-Tau antibody administered to the subject is increased after the third administration. For example, a subject may receive three doses of 750 mg of the anti-Tau antibody every four weeks followed by a dose of 1500 mg every four weeks. A subject may receive three doses of 1500 mg of the anti-Tau antibody every four weeks followed by a dose of 3000 mg every four weeks. According to some embodiments, a subject may receive three doses of 3000 mg of the anti-Tau antibody every four weeks followed by a dose of 4500 mg every four weeks.

The following example is provided to further describe some of the embodiments disclosed herein. The example is intended to illustrate, not to limit, the disclosed embodiments.

### EXAMPLES

### Example 1. A Randomized, Double-Blind, Placebo-Controlled, Combined Single Ascending Dose and Multiple Ascending Dose Study to Assess Safety, Tolerability, Pharmacokinetics, Immunogenicity, and Pharmacodynamics of Intravenous Infusions of E2814 in Healthy Subjects (Study E2814-A001-001)

### Study Rationale and Design:

Study E2814-A001-001 is a randomized, double-blind, placebo-controlled, combined single ascending dose (SAD) and multiple ascending dose (MAD) study to assess safety, tolerability, pharmacokinetics (PK), immunogenicity, and pharmacodynamics (PD) (target engagement [TE]) of intravenous infusions of E2814 in healthy subjects.

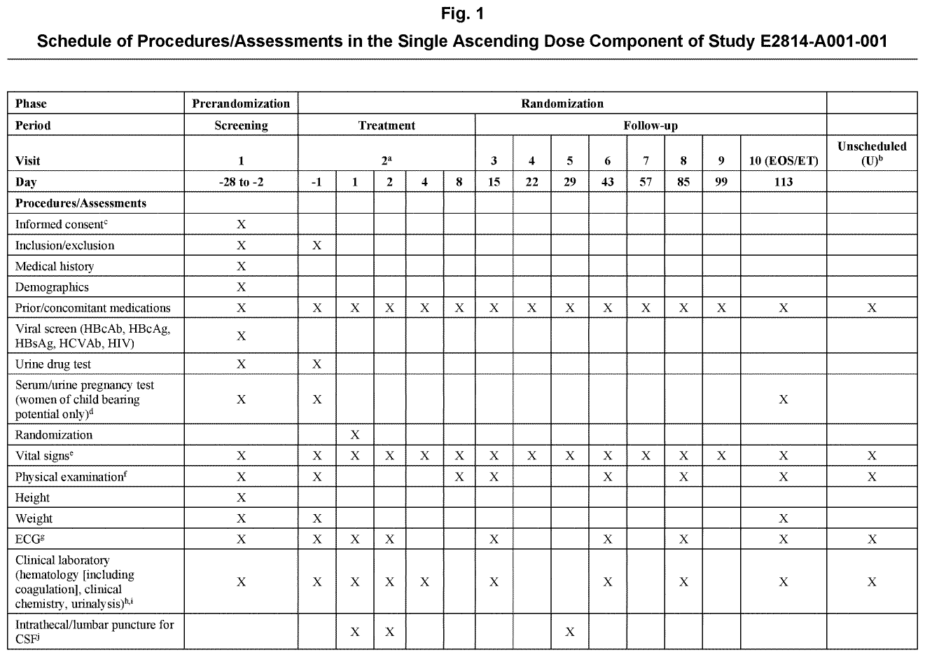

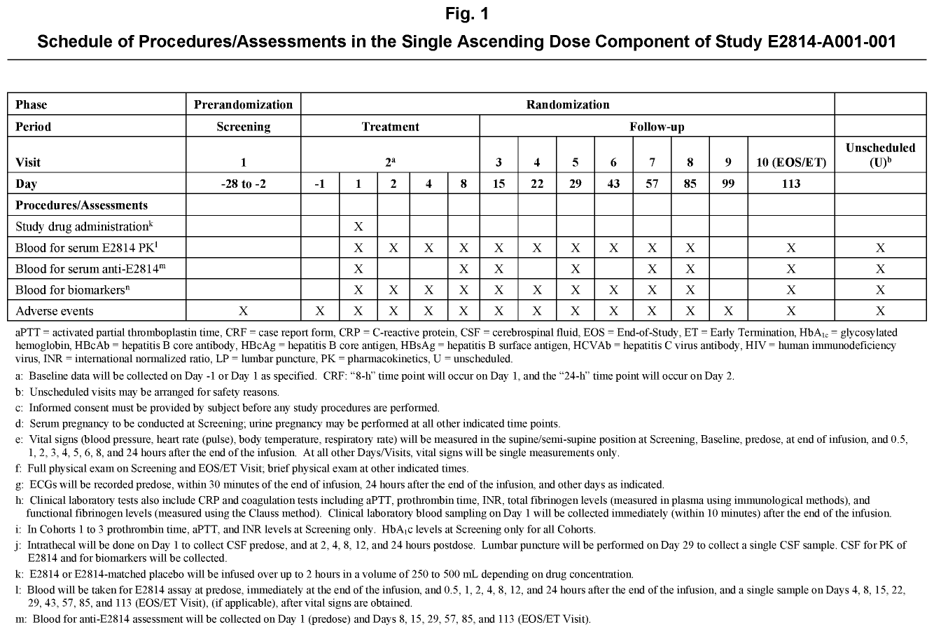

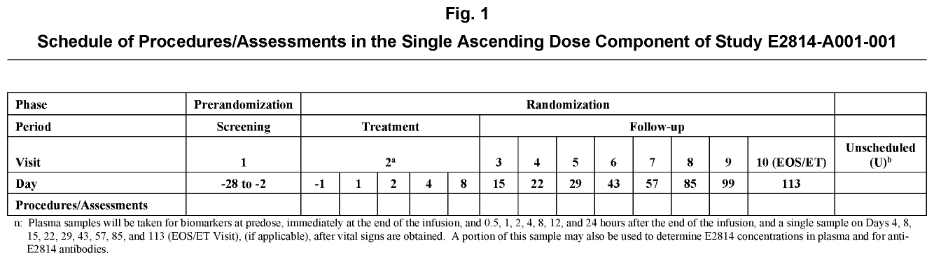

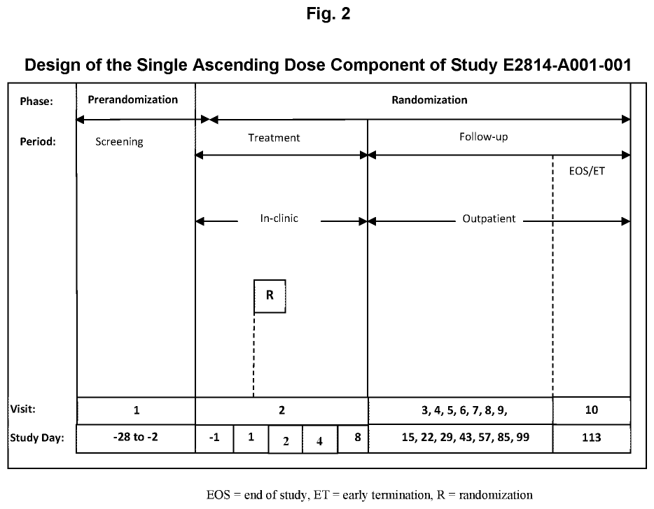

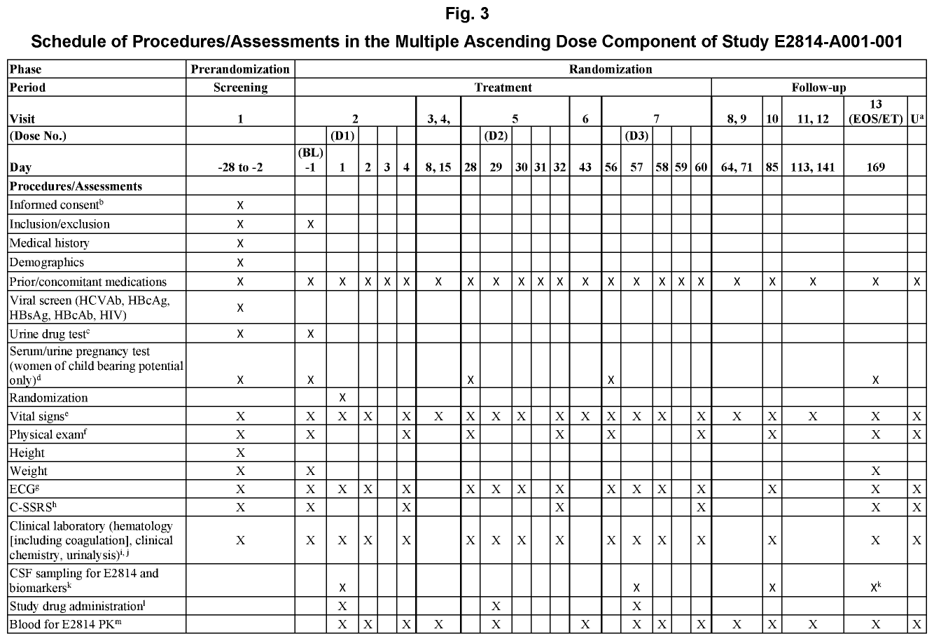

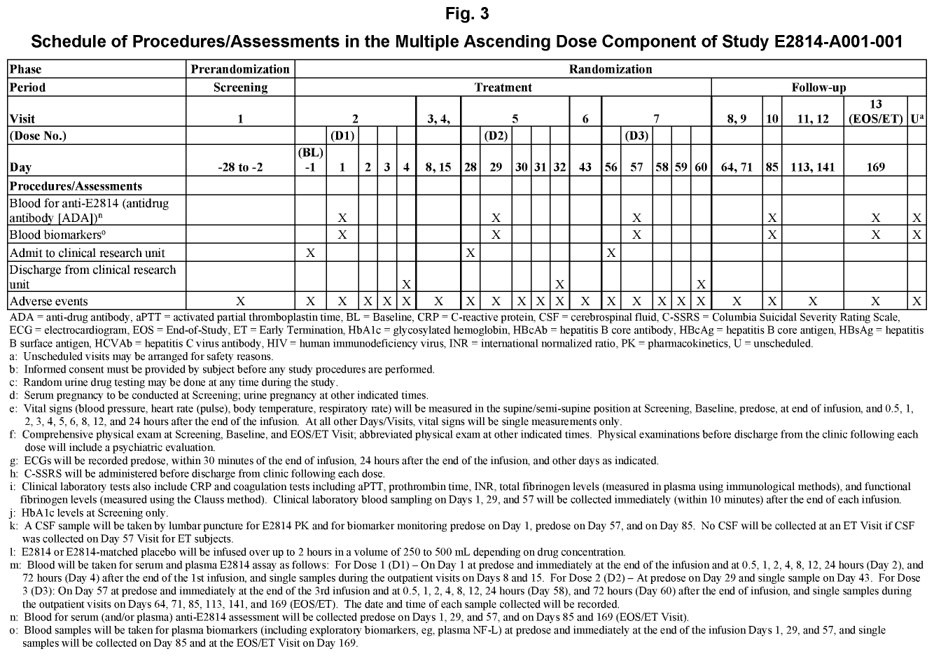

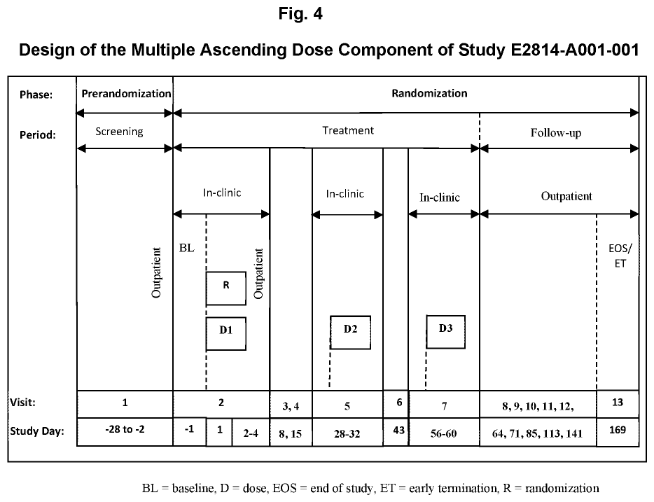

The study is comprised of 2 components:
1) The SAD component evaluates intravenous doses of 3, 10, 30, 60, and 90 mg/kg in healthy subjects to assess safety, tolerability, PK, immunogenicity, and exploratory TE of E2814. The SAD component consists of 2 phases: A Prerandomization Phase (consisting of a Screening Period (Day −28 to Day −2)) and a Randomization Phase (consisting of a Treatment Period and a Follow-Up Period). In the Treatment Period, after Screening procedures are/were completed, Baseline assessments of the subjects were/will be made on Day −1 (FIG. 1). The SAD component consists of 5 dose cohorts (3, 10, 30, 60, and 90 mg/kg); in each cohort 8 subjects are/were randomized (3:1) to receive a single dose of E2814 or E2814-matched placebo (placebo solution matched to E2814).
All subjects will receive/received a single E2814 or E2814-matched placebo intravenous infusion on Day 1. At each dose level, 2 subjects will be randomized on Day 1: 1 subject to receive E2814 and 1 subject to receive placebo. The remaining 6 subjects in each cohort were/will be randomized, and were/will be dosed at least 24 hours later. The end of the SAD component of the study will be the date of the last study assessment for the last subject. An overview of the design for the SAD component of the study is presented in FIG. 2.
2) The MAD component will evaluate 4 fixed doses administered intravenously on 3 occasions every 4 weeks (Q4W) to assess safety, tolerability, PK, and immunogenicity of E2814 after multiple dose intravenous administration in healthy subjects. The doses are 750, 1500, 3000, and 4500 mg (Q4W). The MAD component consists of 2 phases: A Prerandomization Phase (consisting of a Screening Period (Day −28 to Day −2)) and a Randomization Phase (consisting of a Treatment Period and a Follow-Up Period).
In the Treatment Period, after Screening procedures are completed, Baseline assessments of the subjects have been/will be made on Day −1 (FIG. 3). In each cohort (750, 1500, 3000, and 4500 mg (Q4W)), 8 healthy subjects have been/will be randomized (3:1) to receive 3 Q4W doses of E2814 or E2814-matched placebo (placebo solution matched to E2814). The planned dose escalation scheme is 750, 1500, 3000, and 4500 mg. TE was/will be evaluated by measuring free and bound tau species in cerebrospinal fluid (CSF).
The end of the MAD component of the study will be the date of the last study visit assessment for the last subject. An overview of the study design for the MAD component of the study is presented in FIG. 4.

### Study Objectives

The primary objective of the SAD component of the study is to evaluate the safety and tolerability of single intravenous infusions of E2814 in healthy adult subjects. The primary objective of the MAD component of the study is to evaluate the safety and tolerability of 3 Q4W intravenous infusions of E2814 in healthy adult subjects.

The secondary objectives of the SAD component of the study are to assess the PK of E2814 in serum, plasma, and cerebrospinal fluid (CSF); and to assess the immunogenicity (production of serum [or plasma] anti-E2814 antibody) of E2814. The secondary objectives of the MAD component of the study are to assess the PK of E2814 in serum, plasma, and CSF after 3 Q4W intravenous infusions; and to assess the immunogenicity (production of serum [or plasma] anti-E2814 antibody) of E2814 after 3 Q4W intravenous infusions.

The exploratory objectives of both the SAD and MAD components of the study are to compare PK, safety, and tolerability of E2814 between healthy adult non-Japanese and Japanese subjects; to evaluate TE of E2814 on MTBR tau species in CSF; and to explore the effects of E2814 on CSF and/or plasma biomarkers.

### Study Population

Inclusion Criteria. Subjects must meet the following criteria to be included in this study:
1. Nonsmoking, healthy male or female subjects.
2. Age≥20 years and ≤55 years at the time of informed consent

Exclusion Criteria. Subjects who meet any of the following criteria will be excluded from this study:
1. Clinically significant illness that requires medical treatment within 8 weeks or a clinically significant infection that requires medical treatment within 4 weeks of dosing.
2. Females who are breastfeeding or pregnant at Screening or Baseline
3. Females of childbearing potential who:
Within 28 days before study entry, did not use a highly effective method of contraception,
Do not agree to use a highly effective method of contraception throughout the entire study period and for 16 weeks after study drug discontinuation.
4. Males who have not had a successful vasectomy or they and their female partners do not meet the criteria above (i.e., not of childbearing potential or practicing highly effective contraception throughout the study period and for 5 times the half-life of the study drug plus 90 days after study drug discontinuation). If the female partner is pregnant, then males who do not agree to use latex, or synthetic condoms throughout the study period and for 90 days after study drug discontinuation.
5. Evidence of disease that may influence the outcome of the study within 4 weeks before dosing; e.g., psychiatric disorders and disorders of the gastrointestinal tract, liver, kidney, respiratory system, endocrine system, hematological system, neurological system, or cardiovascular system, or subjects who have a congenital abnormality in metabolism
6. Any clinically abnormal symptom or organ impairment found by medical history, physical examinations, vital signs, electrocardiogram (ECG) finding, or laboratory test results that requires medical treatment at Screening or Baseline
7. A prolonged QT (ie, QTc Fridericia interval>450 ms) demonstrated on ECG at Screening or Baseline. A history of risk factors for torsade de pointes (e.g., heart failure, hypokalemia, family history of long QT Syndrome).
8. Persistent systolic blood pressure (SBP)>130 mmHg or diastolic blood pressure (DBP)>85 mmHg at Screening or Baseline.
9. Heart rate less than 45 or more than 100 beats/min at Screening or Baseline.
10. Known history of clinically significant drug allergy at Screening or Baseline
11. Known history of food allergies or presently experiencing significant seasonal or perennial allergy at Screening or Baseline
12. Any history of hypersensitivity reaction to a foreign protein, with clinical features of Grades 2 to 4 as described in National Cancer Institute-Common Terminology Criteria for Adverse Events (CTCAE) Version 5.0, immunoglobulin A (IgA) deficiency, or significant autoimmune disease or disorder.
13. Known to be human immunodeficiency virus (HIV) positive at Screening.
14. Active or chronic (including asymptomatic) viral hepatitis (A, B or C) as demonstrated by positive serology at Screening.
15. History of drug or alcohol dependency or abuse within the 2 years before Screening, or those who have a positive urine drug test or breath (or urine) alcohol test at Screening or Baseline.
16. Intake of over-the-counter medications within 2 weeks before dosing.
17. Currently enrolled in another clinical study or used any investigational drug or device within 30 days (or 5 half-lives, whichever is longer) preceding informed consent
18. Exposure to any biologic drug within 90 days or at least 5 half-lives (whichever is longer), or within 4 weeks for vaccines, before Screening, with the exception of influenza and COVID-19 vaccinations that are allowed up to 7 days before dosing.
19. Engagement in strenuous exercise within 2 weeks before check-in.
20. Any contraindication to continuous CSF sampling via indwelling lumbar catheter or via lumbar puncture (LP)
21. Any history of or current blood clotting or bleeding disorder that is not under adequate control, including a platelet count<50,000, international normalized ratio (INR)>1.3, or partial thromboplastin time (PTT)>upper limit of normal (ULN), or fibrinogen<1.8 g/L or >4.3 g/L at Screening or Baseline. Subjects receiving anticoagulation therapy or identified at risk for hemorrhage.
22. Any lifetime suicidal behavior or psychiatric disease.
23. Any current or prior history of suicidal behavior or psychiatric disease identified by the psychiatrist at the Screening Visit.

### Study Assessments

### 1. Pharmacokinetic (PK) Assessments.

Serum, plasma, and CSF concentrations of E2814 were/will be measured by validated electrochemiluminescence (ECL) assay methods or by a validated immunoprecipitation/purification followed by liquid chromatography with tandem mass spectrometry (LC-MS/MS) methods if available.

SAD: Blood samples for serum and plasma PK assessments were/will be collected predose, immediately at the end of the infusion, and at 0.5, 1, 2, 4, 8, 12, and 24 hours after the end of the infusion, and a single sample each on Days 4, 8, 15, 22, 29, 43, 57, 85, and 113 (EOS/ET Visit) (if applicable). Serum concentrations of E2814 will be analyzed by noncompartmental methods to determine the relevant PK parameters. In addition, In Cohorts 1 through 3 only, blood plasma samples collected for PD biomarkers will also be used to determine E2814 concentrations in plasma. CSF samples for PK/PD assessments will be collected by intrathecal route at predose, 2, 4, 8, 12, and 24 hours after the end of infusion, and a single sample by LP on Day 29.

MAD: Blood samples and CSF samples were/will be collected from each subject as specified in FIG. 3. Blood samples for serum and plasma PK assessments were/will be collected according to the following schedule:
Predose and immediately at the end of the 1st infusion on Day 1, and at 0.5, 1, 2, 4, 8, 12, 24 hours (Day 2), and 72 hours (Day 4) after the end of the infusion. Single samples were/will be collected during the outpatient visits on Days 8 and 15
Predose on Day 29 (2nd infusion) and a single sample on Day 43
Predose and immediately at the end of the 3rd infusion on Day 57, and at 0.5, 1, 2, 4, 8, 12, 24 hours (Day 58), and 72 hours (Day 60) after the end of the infusion. Single samples were/will be collected during the outpatient visits on Days 64, 71, 85, 113, 141, and 169 (End of Study/Early Termination (EOS/ET) Visit) (if applicable).
   Serum and plasma concentrations of E2814 were/will be analyzed by noncompartmental methods to determine the relevant PK parameters.

CSF samples were/will be collected via LP predose on Day 1, predose on Day 57, and on Day 85 for PK and PD assessments.

Pharmacokinetic Analyses. Serum, plasma, and CSF concentrations of E2814 were/will be tabulated by nominal sampling time and summarized by dose using summary statistics. Serum, plasma, and CSF concentration-time profiles were/will be plotted. Using noncompartmental analysis, serum/plasma (SAD and MAD) and CSF concentrations (SAD only) of E2814 were/will be analyzed to determine the PK parameters.

As shown in Table 6, Serum and plasma PK parameters for the SAD and MAD components included/will include (but not be limited to) Cmax, time to reach maximum drug concentration (tmax), AUC(0-24h), AUC(0-72h), terminal elimination half-life (t½), clearance (CL), and volume of distribution (Vz). AUC(0-inf) was/will be estimated for the SAD component only, and AUC(0-tau), ratio of accumulation for Cmax, (Rac(Cmax), and ratio of accumulation for AUC (Rac(AUC)) was/will be estimated for the MAD component only. PK parameters for CSF E2814 include (but are not limited to) Cmax, tmax, and AUC(0-24h). No CSF PK parameters will be estimated for the MAD component.

TABLE 6
PK Parameters

| PK | | Study Component | |
|---|---|---|---|
| parameter | Description | SAD | MAD |
| Cₘₐₓ | maximum observed drug concentration | X | X |
| | | | (Days |
| | | | 1, 57) |
| tₘₐₓ | time to reach maximum (peak) drug | X | X |
| | concentration | | |
| AUC_{(0-24 h)} | area under the concentration-time curve | X | X |
| | from zero time to 24 hours postdose | | (Days |
| | | | 1, 57) |
| AUC_{(0-72 h)} | area under the concentration-time curve | X | X |
| | from zero time to 72 hours postdose | | (Days |
| | | | 1, 57) |
| AUC_{(0-inf)} | area under the concentration-time curve | X | |
| | from zero time extrapolated to infinity | | |
| AUC₍₀₋ₜₐᵤ₎ | area under the concentration-time curve | | X |
| | from zero time to the end of the dosing | | (Days |
| | interval | | 1, 57) |
| t_{1/2} | terminal elimination half-life | X | X |
| | | | (Day 57) |
| CL | clearance | X | X |
| | | | (Day 57) |
| V_{z} | volume of distribution | X | X |
| | | | (Day 57) |
| Rac(Cₘₐₓ) | ratio of accumulation for Cₘₐₓ | | X |
| Rac(AUC) | ratio of accumulation for AUC | | X |

### 2. Pharmacodynamic Assessments

Blood samples and CSF samples were/will be collected as specified in the Schedule of Procedures/Assessments in FIG. 1 for SAD and in FIG. 3 for MAD.

SAD: CSF samples was/will be collected via indwelling intrathecal catheter for establishment of TE analysis approach in humans through measurements of bound MTBR Tau species (e.g., MTBR-Tau354 and MTBR-Tau299) and free MTBR tau species and calculation of total MTBR tau. LP was/will be performed on Day 29. Blood plasma samples for PD biomarkers was/will be collected at predose, immediately at the end of the infusion, and 0.5, 1, 2, 4, 8, 12 and, 24 hours after the end of the infusion, and a single sample on Days 4, 8, 15, 22, 29, 43, 57, 85, and 113 (EOS/ET Visit). Blood plasma samples for PD biomarkers may also be used to determine E2814 concentrations in plasma and for anti-E2814 antibodies, if needed.

MAD: To evaluate TE, CSF samples were/will be collected via LP sampling at predose on Day 1, predose on Day 57, and on Day 85. Blood samples for plasma PD biomarkers were/will be collected at predose and immediately at the end of the infusion on Days 1, 29, and 57. Single samples were/will be collected during outpatient visits on Day 85 and at the EOS/ET Visit on Day 169.

Pharmacodynamic Analyses. Biomarker measurements and change from baseline were/will be summarized by time point and dose and/or treatment group and presented graphically. These analyses were/will be done for both CSF and plasma, as data allow. Dose-response relationships were/will be evaluated as needed. Additional exploratory analyses for biomarkers in CSF and plasma may be performed.

### 3. Pharmacokinetic-Pharmacodynamic Assessments

The PK-PD relationship between E2814 exposure and CSF and/or plasma biomarkers were/will be assessed. This assessment may include, but is not limited to, the characterization of the PK/TE relationship between E2814 concentrations and MTBR tau binding in CSF.

PK-PD Analyses. Data permitting, the relationship between PK and PD was/will be evaluated by visual inspection using plots. This may include, but is not limited to, the graphical exploration of the PK/TE relationship between E2814 concentrations and MTBR tau binding in CSF.

### 4. Safety Assessments

Safety assessments consisted/will consist of monitoring and recording all AEs; regular monitoring of hematology (including coagulation in the SAD [Cohorts 4 and 5] component and all cohorts of the MAD only component), clinical chemistry, and urine values; periodic measurement of vital signs and ECGs; periodic evaluation of suicidality using the C-SSRS (MAD only), and performance of physical examinations (including a psychiatric evaluation) will be performed as detailed in the Schedule of Procedures/Assessments for the SAD component (FIG. 1) and for the MAD component (FIG. 3).

Safety Analyses. Safety data that were/will be evaluated include AEs, clinical laboratory results, vital signs, ECGs, C-SSRS (MAD only), and physical examinations (including a psychiatric evaluation). Descriptive statistics (eg, mean, SD, median, minimum, and maximum for continuous variables, and the number and percent for categorical variables) of the laboratory, vital signs, and ECGs, and changes from baseline were/will be evaluated by dose.

### 5. Laboratory Measurements

Clinical laboratory tests to be performed included hematology (including coagulation in MAD component only), clinical chemistry, and urinalysis. The Schedule of Procedures/Assessments (FIG. 1 for the SAD component and FIG. 3 for the MAD component) shows the visits and time points at which blood for clinical laboratory tests and urine for urinalysis were/will be collected in the study.

### 6. Immunogenicity.

Anti-E2814 antibodies were/will be measured by appropriately validated ECL assay methods. Immunogenicity will be assessed by measuring the presence of anti-E2814 antibodies in serum (and/or plasma) at various time points postdose. In addition, clinical measures to monitor for inflammation that may be associated with immunogenicity were/will be implemented in the study, including close monitoring for changes in white blood cell (WBC)/red blood cell (RBC) counts with differentials and in blood levels of 2 acute phase inflammatory markers, C-reactive protein (CRP), and fibrinogen. Additional safety assessments for subjects suspected of having an immunologic response may include measurements of cytokine responses, lymphocyte counts and subsets, immunoelectrophoresis, or any other clinically appropriate assessments.

The number (percentage) of subjects with positive and negative anti-drug antibodies (ADA) and ADA titer categories (>0, 5, 25, 125, etc) by visit and dose will be summarized. In addition, the correlation between anti-drug antibody (ADA) titer and PK profile was/will be evaluated at the minimum using descriptive statistics and summary plots.

### Study Endpoints

### 1. Primary Endpoint.

The primary endpoint for the SAD and MAD components is the incidence of treatment emergent adverse events (TEAEs) and treatment-emergent serious adverse events (SAEs), laboratory parameters, vital signs, and ECGs.

### 2. Secondary Endpoints

### SAD:

PK parameters derived by noncompartmental analysis using serum, plasma, and CSF concentrations of E2814;
Serum (and/or plasma) anti-E2814 antibody concentration.

### MAD:

PK parameters derived by noncompartmental analysis using serum and plasma concentrations of E2814 following infusion on Days 1 and 57, and the CSF E2814 concentration predose on Day 57 and on Day 85 (ie, 28 days after the 2nd and 3rd infusions, respectively);
Serum (and/or plasma) anti-E2814 antibody concentration.

### 3. Exploratory Endpoints

### SAD and MAD:

Change from baseline in CSF free and bound MTBR tau and total MTBR tau
Change from baseline in CSF and/or plasma biomarkers including t-tau and p-tau

### Analysis Sets:

The Safety Analysis Set was the group of subjects who received at least 1 dose of study drug and had at least 1 post-dose safety assessment. The PK Analysis Set was the group of subjects who received at least 1 dose of study drug and had sufficient PK data to derive at least 1 PK parameter. The PD Analysis Set was the group of subjects who received at least 1 dose of study drug and had sufficient PD data to derive at least 1 PD parameter.

### Preliminary Results: Clinical Safety.

### SAD Component (Study E2814-A001-001):

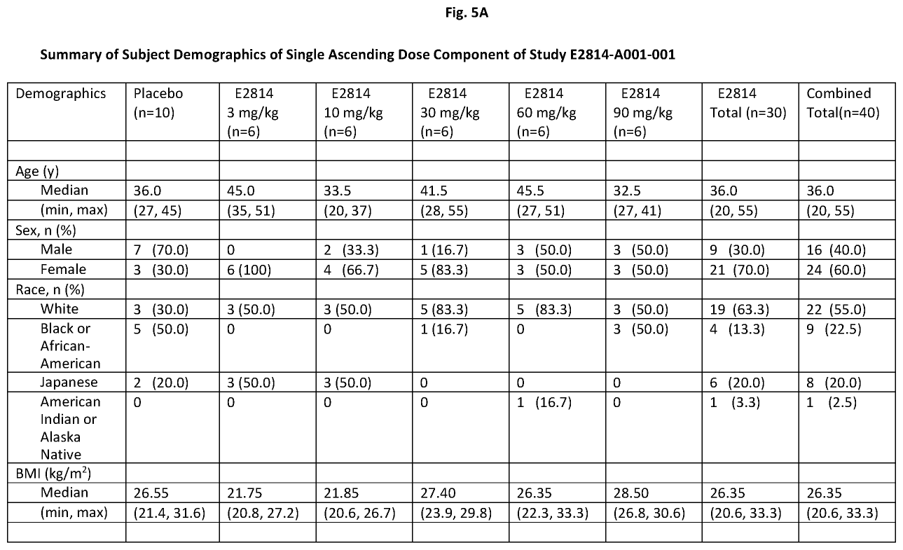

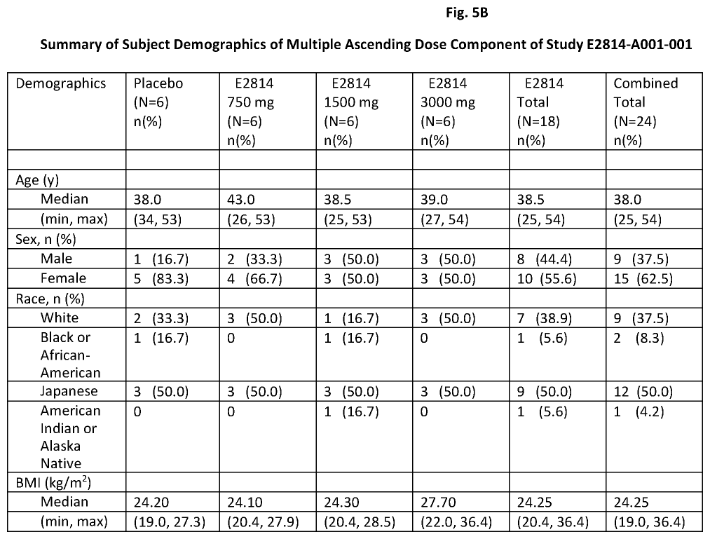

The SAD component of study E2814-A001-001 has completed the evaluation of three cohorts with corresponding E2814 dose levels of 3, 10, and 30 mg/kg. In each cohort, 6 subjects received E2814 and 2 subjects received E2814-matched placebo. A total of 24 healthy subjects have been randomized to the SAD component of study E2814-A001-001, of which 18 were administered E2814 and 6 placebo. All 24 subjects enrolled in the study received at least 1 dose of placebo or E2814 and were included in the safety population. Subject demographics and baseline characteristics are summarized in FIG. 5A.

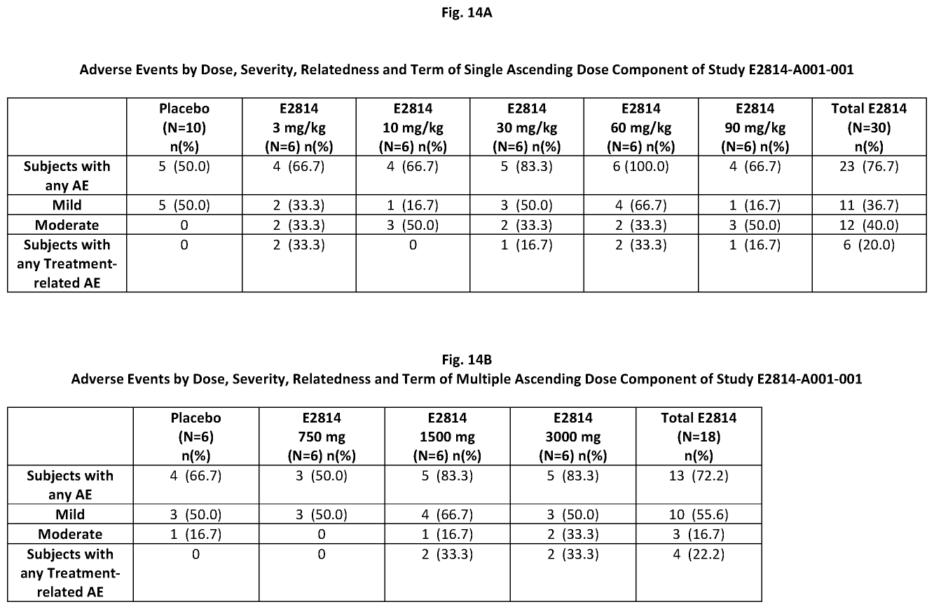

The SAD component safety results (Cohorts 1 to 5; FIG. 14A) demonstrate that E2814 has an adequate safety and tolerability profile as shown by the absence of clinically significant drug-related laboratory, ECG or examination safety findings or dose limiting adverse events (AE) across the evaluated doses of 3, 10, 30, 60, and 90 mg/kg. There were no treatment-emergent serious adverse events or severe AEs. Two AEs, skin rash and headache, both mild in severity, were deemed by investigator to be related to study drug. One subject in cohort 3 had an elevated C-Reactive Protein (CRP) compared to baseline notable on Days 2 and 3 that was asymptomatic and resolved without treatment. The maximum tolerated dose (MTD) was not identified.

Furthermore, in each of Cohort 4 (60 mg/kg) and Cohort 5 (90 mg/kg) to date, six subjects have received E2814 and two subjects have received E2814-matched placebo. The preliminary data from these cohorts demonstrated that there were no treatment-emergent serious adverse events or severe adverse events and no clinically significant findings in vital signs, EEG, and laboratory data.

### MAD Component (Study E2814-A001-001):

Subject demographics and baseline characteristics are summarized in FIG. 5B.

The MAD component of study E2814-A001-001 has evaluated 2 cohorts with corresponding E2814 dose levels of 750 mg and 1500 mg every 4 weeks (Q4W) for a total of 3 doses with data available up to Day 169 (the last study visit) and Day 85, respectively. In each MAD cohort, 6 subjects received E2814 and 2 subjects received E2814-matched placebo. A total of 16 healthy subjects were randomized, of whom 12 subjects received intravenous E2814 (6 active per cohort) and 4 subjects received placebo (2 per cohort). A total of 3 subjects were withdrawn for reasons other than drug-related safety events: 2 subjects were withdrawn after dosing on Day 1 due to preexisting conditions (1 infectious skin rash [750 mg] and 1 asymptomatic M-spike [1500 mg]) that were not identified at the Baseline visit and before study drug dosing. The third subject was withdrawn prior to receiving the third dose, as the dosing visit could not be re-scheduled within the per protocol allowable window. This subject had mild respiratory symptoms (COVID-19 PCR negative), which were deemed not related to the study drug.

The MAD component safety results demonstrate that E2814 has an adequate safety and tolerability profile as shown by the absence of clinically significant drug-related laboratory, vital signs, ECG or physical examination safety findings or dose limiting AE across the evaluated doses of 750 and 1500 mg Q4W. In total, 8 of 12 subjects (66.7%) treated with E2814 and 2 of 4 subjects (50.0%) treated with placebo experienced at least 1 TEAE during the study. There were no treatment-emergent serious AEs or severe AEs. All abnormal laboratory findings were not associated with symptoms in subjects and were considered not clinically significant by the Principal Investigator. Across all subjects in the MAD cohorts treated with E2814, the most common TEAEs were headache (16.7%, 2 subjects), back pain (16.7%, 2 subjects), and skin rash (16.7%, 2 subjects). The maximum tolerated dose (MTD) was not identified.

Furthermore, in Cohort 3 (3000 mg Q4W) to date, five subjects have received E2814 and two subjects have received E2814-matched placebo in the cohort. The preliminary data from the cohort also demonstrated doses of 3000 mg Q4W E2814 had an acceptable safety and tolerability profile in healthy volunteers. Treatment-emergent adverse events (TEAEs) were generally mild in intensity, with three moderate (2 headaches [1 related], 1 nausea [related]). There were no treatment-emergent serious adverse events or severe adverse events and no clinically significant findings in vital signs, EEG, and laboratory data. Safety data for the MAD component of study E2814-A001-001 is summarized in FIG. 14B.

Safety results from the SAD and MAD component of study E2814-A001-001 demonstrate that E2814 has an adequate single and multiple dose safety and tolerability profile, with no clinically significant drug-related laboratory, coagulation parameters (fibrinogen, INR, PT, aPTT), vital sign, ECG or physical examination safety findings or dose limiting adverse events (AE) across the evaluated cohorts. There were no dose-limiting events (DLEs) at doses up to the highest evaluated dose of 90 mg/kg and 3000 mg Q4W in the SAD and MAD components, respectively.

### Preliminary Results: Clinical Pharmacology

The single dose PK and TE of E2814 have been investigated in a total of 18 healthy adult male and female subjects in the SAD component of study E2814-A001-001. The PK and TE following 3 repeat Q4W infusions have been evaluated in a total of 12 subjects in the MAD component of the same study.

### Preliminary Results: Clinical Pharmakinetics (PK)

### SAD Component—PK and ADA (Study E2814-A001-001)

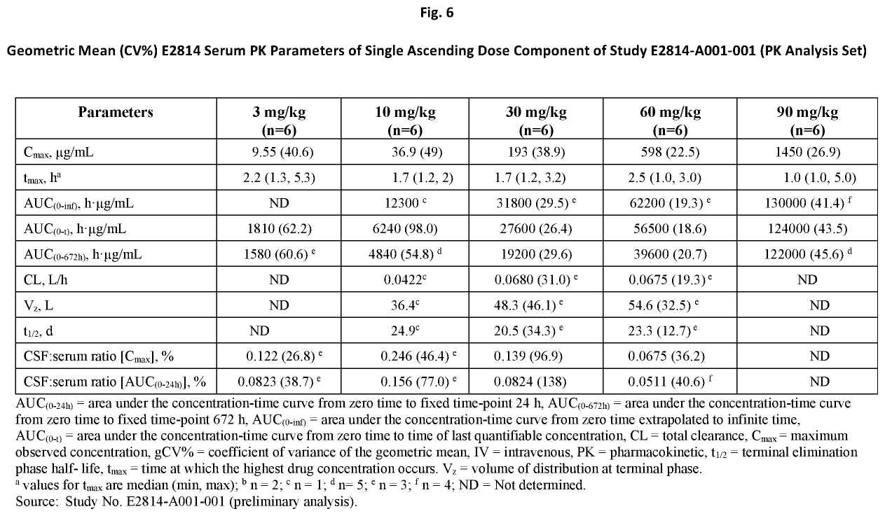

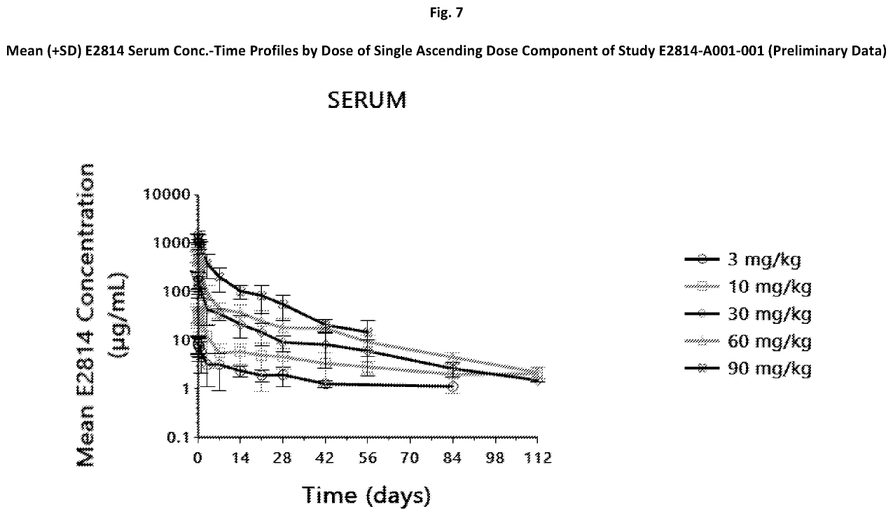

Mean serum concentration-time profiles of E2814 following single intravenous dose administration in the SAD component are shown in FIG. 7. The geometric mean serum PK parameters are presented in FIG. 6.

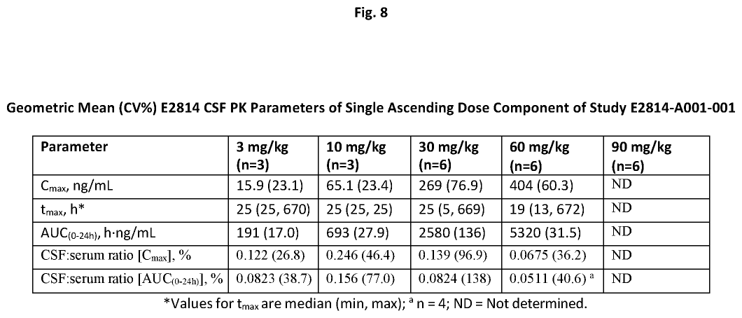

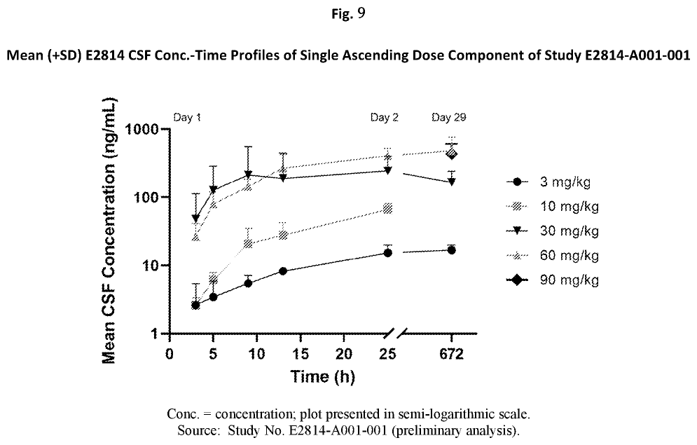

PK results indicate there was a dose-related increase in serum E2814 exposures (FIG. 6). The observed serum AUC and Cₘₐₓ were approximately dose proportional from 3 to 30 mg/kg and greater than dose proportional at higher doses of 60 mg/kg and 90 mg/kg (FIG. 6). The median time to maximum E2814 concentrations in serum (tmax) was 1 to 2.5 hours. Across dose groups, E2814 presented a range of volume of distribution (Vz) of ˜36 L to 55 L, a clearance (CL) of 0.04 to 0.07 L/hour, and a half-life (t_{1/2}) of 20 to 25 days. The serum-to-CSF concentration ratio ranged between 0.1% to 0.3% (FIG. 6). Mean CSF concentration-time profiles of E2814 are shown in FIG. 9. The geometric mean CSF PK parameters are presented in FIG. 8. CSF PK results indicate a greater than dose proportional increase in Cₘₐₓ and AUC (0-24h) between the dose range of 3 to 60 mg/kg, with a median tmax of 25h across these dose groups.

The presence of anti-E2814 antibodies (ADA) in serum was confirmed in 8 out of 24 E2814-treated subjects (3 subjects in the 3 mg/kg cohort, 1 subject in the 30 mg/kg cohort, and 4 subjects in the 60 mg/kg cohort). Of the total 8 subjects, 5 subjects had transient low-level serum anti-E2814 antibody titers by Day 113 (end of study, EOS). All positive subjects, with the exception of one in the 3 mg/kg cohort, returned to baseline status during the follow-up. The observed E2814 pharmacokinetics in the ADA positive subjects was comparable to that of ADA negative subjects.

### MAD Component—PK and ADA (Study E2814-A001-001)

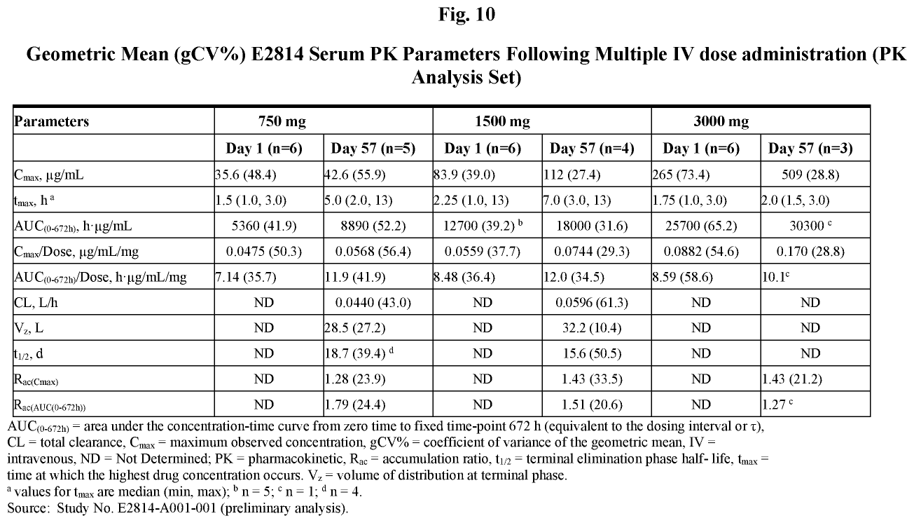

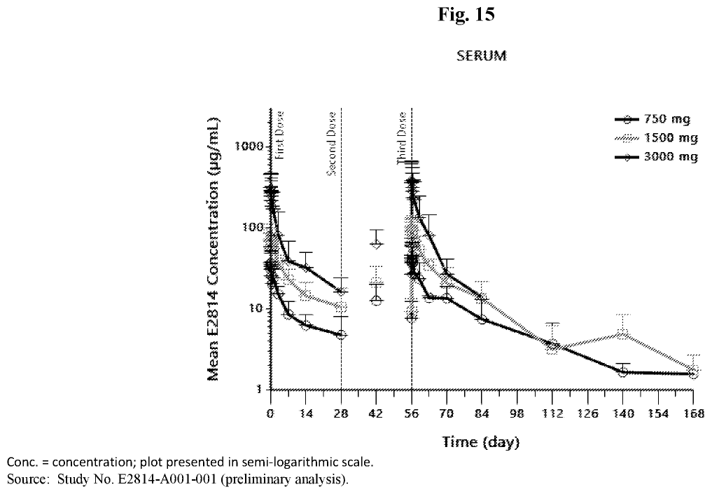

Mean serum concentration-time profiles of E2814 following multiple intravenous dose administration in the MAD component are shown in FIG. 15. The geometric mean serum PK parameters are presented in FIG. 10. The E2814 concentration-time profiles after dosing on Day 1 and Day 57 show that serum concentrations peaked shortly after the end of each infusion. There was a dose-related increase in E2814 concentrations in serum over the three investigated MAD doses. The median time to maximum E2814 concentrations in serum (tmax) was 1.5 to 2.25 hours on Day 1, with an apparent delay in the median tmax values following the 3rd infusion (5 to 7 hours). The observed Day 1 and Day 57 serum geometric mean AUC values increased in a dose proportional manner. The increase in Cₘₐₓ appeared to be more than dose-proportional. E2814 presented a range of volume of distribution (V_{z}) of ˜28-32 L, clearance (CL) of ˜0.04-0.06 L/hour, and a half-life (t_{1/2}) in serum of ˜16-19 days, which was approximately comparable to the observed SAD component values. The accumulation ratio ranged between 1.28 to 1.43 for Cₘₐₓ, and 1.27 to 1.79 for AUC₍₀₋₆₇₂ₕ₎.

Serum ADA were evaluated in the 750 mg and 1500 mg dose groups. Only one subject in the 750 mg dose group had a confirmatory positive prior to dose on Day 1.

### Preliminary Results: Target Engagement in Humans

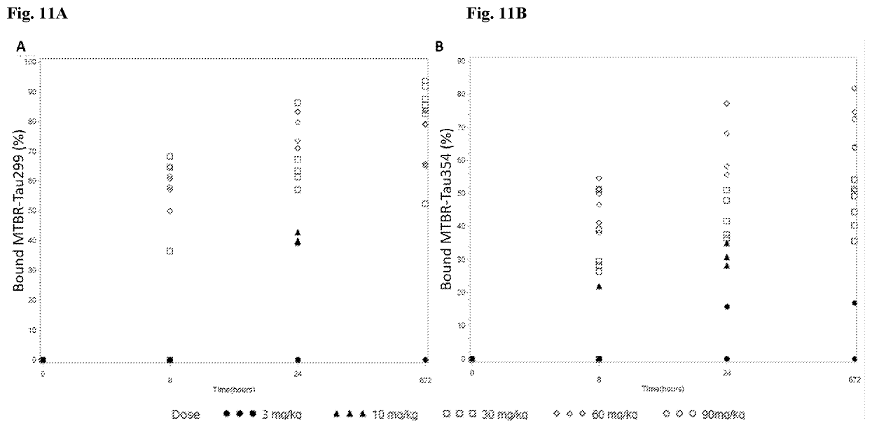

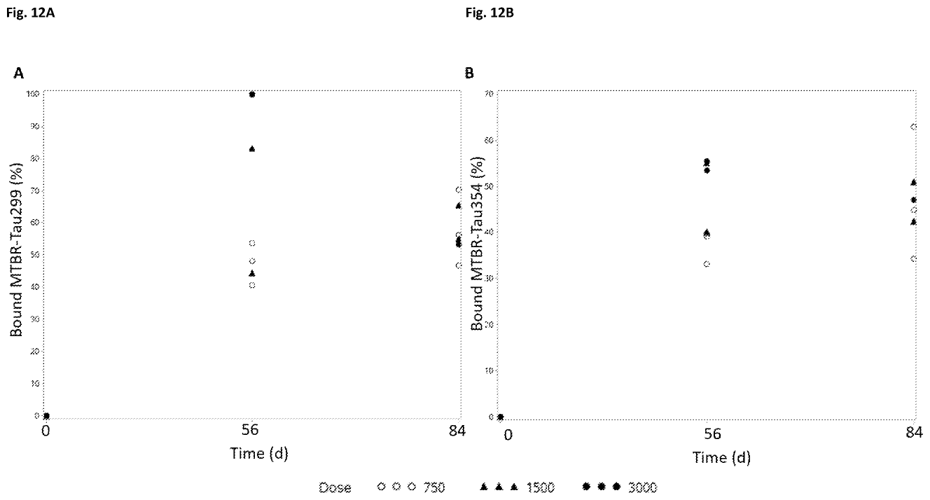

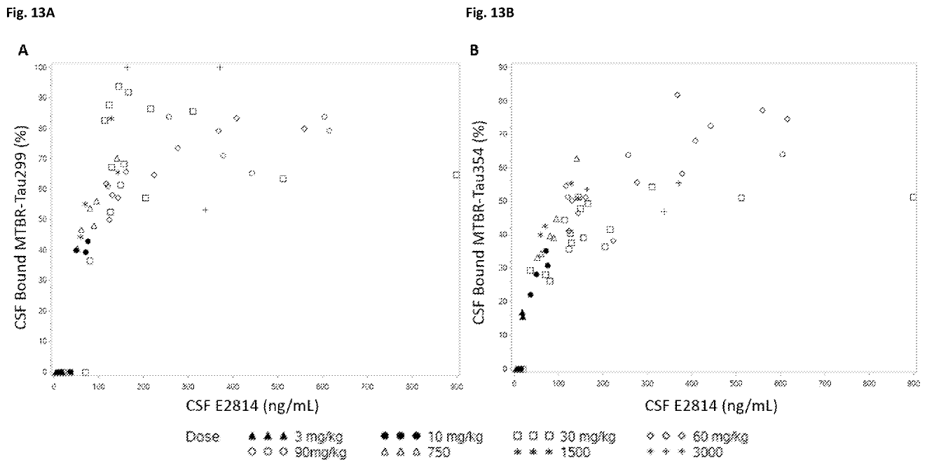

Target engagement (TE) was evaluated in CSF by measuring E2814-bound and free MTBR-tau proxy peptide concentrations (MTBR-tau354 and MTBR-tau299 containing epitopes in R4 and R2, respectively). Preliminary data in healthy subjects indicate that, following administration of E2814, there is an E2814 concentration-related increase in bound MTBR-tau and a decrease in free MTBR-tau levels in CSF following single 3-90 mg/kg administration and multiple 750 mg, 1500 mg, and 3000 mg administrations. Target engagement was calculated as the ratio of E2814-bound MTBR-tau to total (free plus bound) MTBR-tau expressed as a percent. Target engagement levels appeared to be sustained from 24 hours to −672 hours (28 days) following single dose administration (FIGS. 11A and 11B) and from Day 56 to Day 84 following multiple dose administration (FIGS. 12A and 12B). Based on the available CSF TE data from study E2814-A001-001 in healthy volunteers, the highest multiple dose 3000 mg with CSF concentrations ranging between 200-400 ng/mL appears to be saturating binding ˜80% to MTBR-tau299 and ˜70% MTBR-tau354 (FIGS. 13A and 13B).

### Example 2. An Open-Label Phase 1b/2 Study to Assess Safety and Target Engagement of E2814 in Subjects with Mild to Moderate Cognitive Impairment Due to Dominantly Inherited Alzheimer's Disease (Study E2814-G000-103)

### Study Rationale and Study Design

This study is an open-label Phase 1b/2 study to evaluate the safety and target engagement (TE) of 2 different doses of E2814 following intravenous (IV) infusion on MTBR-tau species in cerebrospinal fluid (CSF) in subjects with Dominantly Inherited Alzheimer's Disease (DIAD) and exhibiting mild to moderate cognitive impairment. This study will target individuals who are known to have a disease-causing mutation confirmed by the genetic testing. Subjects in this study are confirmed mutation positive for genes known to be associated with DIAD. The mutations in presenilin 1 (PSEN1), presenilin 2 (PSEN2) and amyloid precursor protein (APP) that are associated with DIAD have very high penetrance (near 100%).

The study will also assess the pharmacokinetic (PK), immunogenicity, and other pharmacodynamic (PD) effects of E2814.

The study will consist of 2 phases: A Pretreatment Phase consisting of a Screening Period (between Day −60 and Day −2; screening assessments will include tau PET, amyloid PET, and safety magnetic resonance imaging (MRI) and genetic testing to confirm mutation status.) and a Treatment Phase consisting of 3 periods: 1b, 2, and follow up.

Phase 1b Treatment Period. The Phase 1b Treatment Period will initially allow 8 subjects to receive open-label treatment with 3 IV infusions of 750 mg E2814 every 4 weeks (Q4W) over 12 weeks. On conclusion of the Phase 1b Treatment Period, on Day 84, subjects will undergo safety assessments. A CSF sample will be collected for assessment of TE and CSF concentrations of E2814 on Day 1 and Day 84. Subjects will then progress to the Phase 2 Treatment Period.

Phase 2 Treatment Period. The Phase 2 Treatment Period will allow subjects who tolerated the 750 mg dose of E2814 and completed all assessments in the Phase 1b Treatment Period to receive a further 96 weeks of IV E2814 at an initial dose of 1500 mg Q4W for at least 3 doses (12 weeks) followed by a dose of 3000 mg Q4W for the remaining weeks.

Following Day 86, all subsequent visits will occur every 4 weeks for the duration of the study. A CSF sample will be collected for assessment of TE and CSF concentration of E2814 on Day 84 (Week 12); thereafter, lumbar puncture (LP) sampling will occur 12 weeks after each dose titration, on Day 169 (Week 24, i.e., 12 weeks after initiation of the 1500 mg dose), Day 253 (Week 36, i.e., 12 weeks after initiation of 3000 mg dose [this collection can occur on a later study day if the increase to 3000 mg dose occurs later than Day 169]) and on Days 421 (Week 60) and 757 (Week 108) for assessment of biomarker and PK endpoints. Subjects will also undergo yearly tau PET scans and amyloid PET scans (3 times in the entire study: Screening, Day 421, and Day 757) and half-yearly assessments of cognitive performance. For subjects who discontinue early, an early termination CSF collection and PET scan will be performed, unless a PET assessment was performed within the previous 3 months.

Follow-Up Period. Subjects will be followed for a period of 12 weeks after the last dose for safety.

The end of the study will be the date of the last study visit for the last subject in the study.

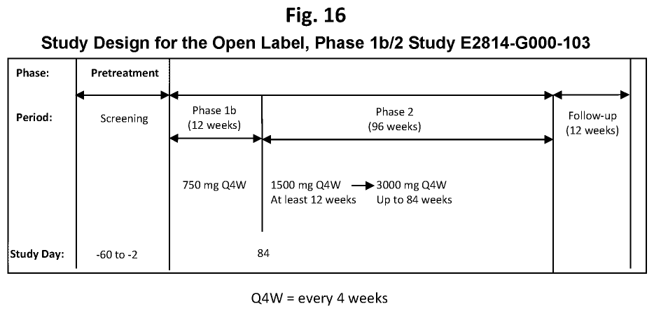

An overview of the study design is presented in FIG. 16.

### Study Objectives

The primary objectives of the study are:
To assess the safety and tolerability of intravenous (IV) infusions of E2814 in subjects with DIAD;
To evaluate TE of E2814 on MTBR-tau species in CSF in subjects with DIAD.
   The secondary objectives of the study are:
To assess the PK of E2814 in serum, plasma, and CSF;
To assess the immunogenicity (production of anti-E2814 antibody) of E2814;
To assess the effect of E2814 on CSF, blood, and imaging biomarkers.

The exploratory objectives of the study are:
To assess the effects of E2814 on the clinical progression of DIAD as assessed using clinical tests such as Clinical Dementia Rating-Sum of Boxes (CDR-SB) and multiple cognitive and clinical endpoints;
To collect genomic samples for potential exploratory investigation on heterogeneity in drug-response and clinical features of disease.

### Study Population

Inclusion Criteria. Subjects must meet all of the following criteria to be included in this study:
1. Male or female, age 18 to 80 years at the time of informed consent
2. Individuals who are confirmed to be mutation positive for PSEN1, APP, or PSEN2 gene that is associated with DIAD
3. Clinical Dementia Rating-Sum of Boxes (CDR-SB) score 5 to 12 at Screening
4. Evidence of positive amyloid status based on historical or screening amyloid PET
5. Able to undergo MRI, LP, positron emission tomography (PET), and complete all study-related testing and evaluations
6. Has a study partner who in the investigator's judgment is able to provide accurate information as to the subject's cognitive and functional abilities, who agrees to provide information at the study visits which require informant input for scale completion

Exclusion Criteria. Subjects who meet any of the following criteria will be excluded from this study:
1. Clinically significant illness that required medical treatment within 8 weeks before the first dose or a clinically significant infection that required medical treatment within 4 weeks before first dose
2. Females who are breastfeeding or pregnant at Screening or Baseline
3. Females of childbearing potential who, within 3 months before screening, did not use a highly effective method of contraception or do not agree to use a highly effective method of contraception throughout the entire study period and for 16 weeks after study drug discontinuation.
4. Any neurological condition that may be contributing to cognitive impairment above and beyond that caused by the subject's AD
5. History of transient ischemic attacks, stroke, or seizures within 12 months of Screening
6. History of clinically important carotid or vertebrobasilar stenosis, plaque, or other prominent risk factor for stroke or cerebral haemorrhage (including atrial fibrillation and anticoagulation).
7. Any current psychiatric diagnosis or symptoms, (eg, hallucinations, major depression, or delusions) that could interfere with study procedures in the subject
8. Geriatric Depression Scale (GDS) score greater than or equal to 8 at Screening
9. Contraindications to MRI scanning, including but not limited to pacemaker/cardiac defibrillator, neurostimulators, ferromagnetic metal implants (eg, in skull and cardiac devices other than those approved as safe for use in MRI scanners)
10. Evidence of other clinically significant lesions on brain MRI at Screening that could indicate a dementia diagnosis other than AD
11. Other significant pathological findings on brain MRI at Screening, including but not limited to the following: more than 15-20 microhemorrhages (defined as 10 mm or less at the greatest diameter); any macrohemorrhage (greater than 10 mm at greatest diameter) which is currently symptomatic at Screening; any area of superficial siderosis which is currently symptomatic at Screening, evidence of vasogenic edema; evidence of cerebral contusion, encephalomalacia, aneurysms, vascular malformations, or infective lesions; evidence of more than one lacunar infarct lacunar infarcts or stroke involving a major vascular territory, severe small vessel, or white matter disease; space occupying lesions; or brain tumors (however, lesions diagnosed as meningiomas or arachnoid cysts and less than 1 cm at their greatest diameter need not be exclusionary)
12. Hypersensitivity to E2814 or any of the excipients, or to any mAb treatment
13. Any immunological disease which is not adequately controlled, or which requires treatment with immunoglobulins, systemic monoclonal antibodies (or derivatives of monoclonal antibodies), systemic immunosuppressants, or plasmapheresis during the study
14. With a bleeding disorder of current chronic use of anticoagulants (eg, warfarin, dabigatran, rivaroxaban or apixaban) or of clopidogrel is exclusionary. Limited (occasional or isolated) use of anticoagulants/antiplatelet compounds in cases such as surgical procedures.
15. Have thyroid stimulating hormone outside of normal range. Other tests of thyroid function with results outside the normal range should only be exclusionary if they are considered clinically significant by the investigator. This applies to all subjects whether or not they are taking thyroid supplements.
16. HgbA1c>8% (retesting is permitted if slightly elevated) or poorly controlled insulin-dependent diabetes (including hypoglycemic episodes). Subjects may be rescreened after 3 months to allow optimization of diabetic control.
17. Abnormally low serum vitamin B12 levels for the testing laboratory (if subject is taking vitamin B12 injections, level should be at or above the lower limit of normal [LLN] for the testing laboratory). Levels of vitamin B12 may be confirmed with reflex testing to include methylmalonic acid analysis, if available in region.
18. History of human immunodeficiency virus (HIV) infection, history of hepatitis B infection within the past year, history of hepatitis C infection which has not been adequately treated, or history of spirochete infection of the central nervous system (eg, syphilis, Lyme, or borreliosis)
19. Any other clinically significant abnormalities in physical examination, vital signs, laboratory tests, or ECG at Screening or Baseline which in the opinion of the investigator require further investigation or treatment or which may interfere with study procedures or safety
20. Malignant neoplasms within 3 years of Screening (except for basal or squamous cell carcinoma in situ of the skin, or localized prostate cancer in male subjects, or localized breast cancer in female subjects). Subjects who had malignant neoplasms but who have had at least 3 years of documented uninterrupted remission before Screening need not be excluded.
21. Answers “yes” to Columbia-Suicide Severity Rating Scale (C-SSRS) suicidal ideation Type 4 or 5, or any suicidal behavior assessment within 6 months before Screening, at Screening, or at the Baseline Visit, or has been hospitalized or treated for any suicidal behavior in lifetime.
22. Known or suspected history of drug or alcohol abuse or dependence within 2 years before Screening or a positive urine drug test at Screening. Subjects who test positive for benzodiazepines or opioids in urine drug testing need not be excluded if in the clinical opinion of the investigator, this is due to the subject taking prior/concomitant medications containing benzodiazepines or opioids for a medical condition and not due to drug abuse.
23. Any other medical conditions (eg, cardiac, respiratory, gastrointestinal, renal disease) which are not stably and adequately controlled, or which in the opinion of the investigator could affect the subject's safety or interfere with the study assessments
24. Concurrent participation in a clinical study involving any anti-amyloid therapies (including any mAb therapies) within 6 months before Screening
25. Concurrent participation in a clinical study involving any anti-tau therapies.
26. Participated in any other investigational medication or device study in the 3 months or 5 half-lives (whichever is longer) of the medication before Screening
27. Planned surgery which requires general anesthesia that would take place during the study.
28. Visual or hearing impairment that would prevent the subject from performing psychometric tests accurately.

### Study Assessments

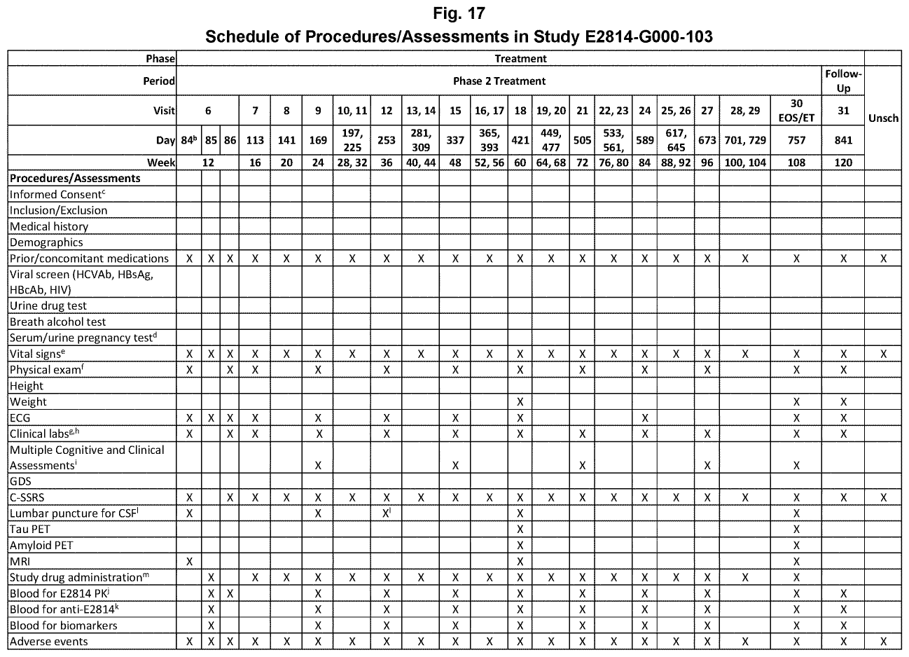

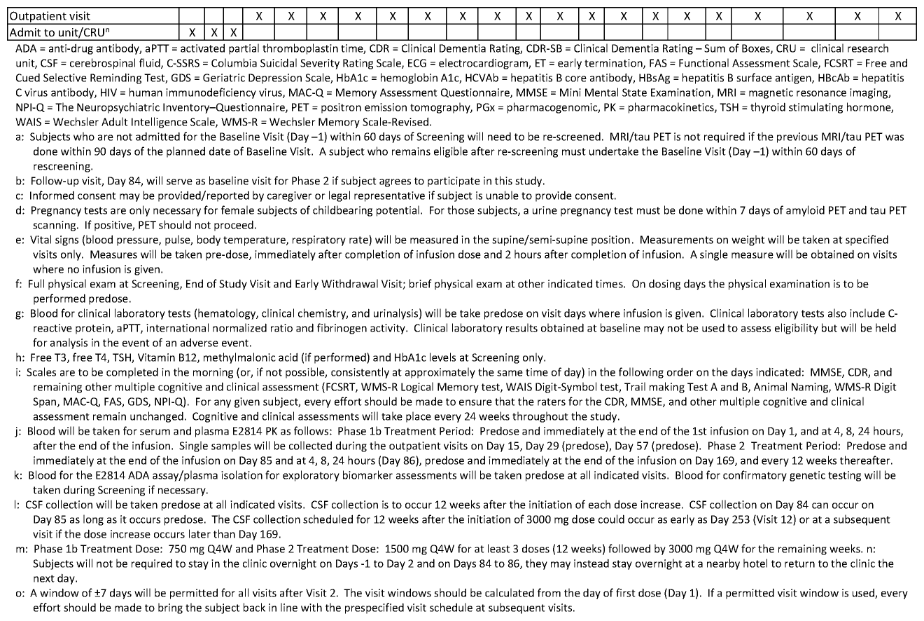

Screening Assessments. Screening assessments will be conducted as specified in the Schedule of Procedures/Assessments (FIG. 17). Subject demography information (age, sex, race/ethnicity) will be collected at the Screening Visit. Medical and surgical history and current medical conditions will be recorded at the Screening Visit. A sample of blood will be taken for hepatitis B core antibody (HBcAb), hepatitis B surface antigen (HBsAg), hepatitis C virus antibody (HCVAb) and HIV tests at the Screening Visit.

Clinical Efficacy Assessments and Analyses. Cognitive assessments will be conducted at baseline (Day −1), every 24 weeks throughout the study, and at the End of Study Visit. These assessments will include global Clinical Dementia Rating (CDR), CDR-SB, Mini Mental State Examination (MMSE), and multiple cognitive and clinical endpoints. The multiple cognitive and clinical endpoints include the following tests: Free and Cued Selective Reminding Test (FCSRT), Wechsler Memory Scale-Revised (WMS-R) Logical Memory, Wechsler Adult Intelligence Scale-Revised (WAIS-R) Digit-Symbol, Trail making Test A and B, Animal Naming, WMS-R Digit Span, Memory Assessment Questionnaire (MAC-Q), Functional Assessment Scale (FAS), GDS, the Neuropsychiatric Inventory-Questionnaire (NPI-Q). Change from baseline in cognitive assessments will be summarized by visit.

Pharmacokinetic (PK) Assessments. Blood samples for PK assessments will be collected according to the following schedule in the Phase 1b Treatment Period (750 mg E2814 administration):
Predose and immediately at the end of the 1st infusion on Day 1, and at 4, 8, and 24 hours after the end of the infusion
Single samples will be collected during the outpatient visits on Day 15, Day 29 (predose), and Day 57 (predose).
   The following PK samples will be taken during the Phase 2 Treatment Period (1500 mg and 3000 mg E2814 administration):
Predose and immediately at the end of the infusion on Day 85 and at 4, 8, and 24 hours after the end of the infusion, then predose and immediately at the end of the infusion on Day 169, and every 12 weeks thereafter.
   Serum and plasma concentrations of E2814 will be analyzed by noncompartmental methods to determine the relevant PK parameters.

CSF samples will be collected via LP predose on Day 1, Day 84 (Week 12 [this collection can occur on Day 85 as long as it occurs predose]). Thereafter, LP sampling will occur 12 weeks after each dose titration; for example, Day 169 (12 weeks after initiation of the 1500 mg dose), Day 253 (12 weeks after initiation of 3000 mg dose [this collection can occur on a later study day if the increase to 3000 mg dose occurs later than Day 169]), Day 421, and Day 757 for PK and pharmacodynamic (PD) assessments.

Serum, plasma, and CSF concentrations of E2814 will be measured by validated electrochemiluminescence assays and/or by a validated immunoprecipitation/purification followed by liquid chromatography with tandem mass spectrometry, if available. Anti-E2814 antibodies will be measured by a validated electrochemiluminescence assay.

Serum and plasma concentrations of E2814 will be tabulated by nominal sampling time and summarized by dose using summary statistics. Serum and plasma concentration-time profiles will be plotted. Serum and plasma E2814 PK parameters will include (but not be limited to) Cₘₐₓ, time to reach maximum drug concentration (tmax) and area under the concentration-time curve from zero time to the end of the dosing interval (AUC₍₀₋₆₇₂ₕ₎) on Days 1 and 85. An integrated population analysis of E2814 PK will be performed by pooling data from all available studies.

Pharmacodynamic Assessments. CSF samples will be collected via LP predose on Day 1 and Day 84 (Week 12 [this LP can take place on Day 85 as long as it is predose]). Thereafter, LP sampling will occur 12 weeks after each dose titration; for example, predose on Day 169 (12 weeks after initiation of the 1500 mg dose), Day 253 (12 weeks after initiation of 3000 mg dose [this collection can occur on a later study day if the increase to 3000 mg dose occurs later than Day 169]) to evaluate MTBR-tau TE (free/bound MTBR-tau species) and CSF concentration of E2814, followed by annual CSF collections on Day 421 (Week 60) and Day 757 (Week 108) for assessment of biomarker and PK endpoints. Blood samples for plasma PD biomarker will be collected predose and immediately at the end of the infusion on Days 1, 29, 57, and 85. Blood samples for plasma PD biomarkers will also be taken on Day 15, Day 169 and every 12 weeks thereafter during the Phase 2 Treatment Period.

Biomarker measurements (fluid and imaging) and change from baseline will be summarized by time point and dose and presented graphically. These analyses will be done for CSF, plasma and serum. Dose-response relationships will be evaluated.

The relationship between PK and PD will be evaluated by visual inspection using plots. This may include but is not limited to the graphical exploration of the PK/TE relationship between E2814 concentrations and MTBR-tau binding in CSF.

Pharmacogenomic (PGx) Assessments. A PGx blood sample for confirmatory PSEN1, APP, or PSEN2 gene mutation testing will be taken during screening. CSF and Blood Plasma Biomarkers. CSF and blood plasma concentrations of AD-related biomarkers (including but not limited to Aβ40, Aβ42, neurogranin, neurofilament light chain, total tau [t tau], and phosphorylated tau biomarkers) will be measured.

Imaging Biomarkers. Longitudinal tau (MK-6240) and amyloid PET (eg, C-Pittsburgh Compound-B or NAV4694) will be performed at Screening and then annually during the Phase 2 Treatment Period. Early termination PET scans will be performed unless a PET assessment was performed within the previous 3 months.

Safety Assessments. For both the Phase 1b and Phase 2 Treatment Periods, safety assessments will consist of monitoring and recording all adverse events (AEs); regular monitoring of hematology, clinical chemistry, and urine values; periodic measurement of vital signs and ECGs; periodic evaluation of suicidality using the C-SSRS, and performance of physical examinations. Safety MRIs will be conducted at Screening, on completion of the Phase 1b Treatment Period, and then annually during the Phase 2 Treatment Period.

Safety data that will be evaluated include AEs, clinical laboratory results, vital signs, ECGs, C-SSRS, and physical examinations. Safety data from the Phase 1b and Phase 2 Treatment Periods will be summarized separately. TEAEs will be summarized by dose. Descriptive statistics (eg, mean, SD, median, minimum, and maximum for continuous variables, and the number and percent for categorical variables) of the laboratory, vital signs, and ECGs, and changes from baseline will be evaluated by dose.

Immunogenicity Assessments. Immunogenicity will be assessed by measuring the presence of anti-E2814 antibodies predose on Days 1, 15, 29, 57, 85, 113, 169, and every 12 weeks during the Phase 2 Treatment Period. In addition, clinical measures to monitor for inflammation that may be associated with immunogenicity will be implemented in the study. These assessments will include close monitoring for changes in white blood cell/red blood cell counts with differentials and in blood levels of 2 acute phase inflammatory markers: C-reactive protein, and fibrinogen. Additional safety assessments for subjects suspected of having an immunologic response may include measurements of cytokine responses, lymphocyte counts and subsets, immunoelectrophoresis, or any other clinically appropriate assessments.

### Statistical and Analytical Plans

### Primary Endpoints:

Incidence of treatment-emergent adverse events (TEAEs) and SAEs, laboratory parameters, vital signs, and ECGs
Change from baseline in CSF free and bound MTBR-tau and total MTBR-tau at 12 weeks

### Secondary Endpoints:

Serum and plasma PK parameters following dosing on Days 1 and 85
CSF E2814 concentrations
Serum (or plasma) anti-E2814 antibody concentration
Change from baseline in CSF and/or plasma biomarkers including total tau (t-tau) and phosphorylated tau biomarkers
Change from baseline in tau PET signal

### Exploratory Endpoints:

Change from baseline in cognitive and clinical assessments
Change from baseline in amyloid PET signal

### Definitions of Analysis Sets

The Safety Analysis Set is the group of all allocated subjects who received at least 1 dose of study drug. At least 1 laboratory, vital sign, or ECG measurement obtained subsequent to at least 1 dose of study treatment is required for inclusion in the analysis of each specific parameter. To assess change from baseline, a baseline measurement is also required. This is the analysis population used for all safety analyses which will be based on as-treated principle.

The PK Analysis Set is the group of subjects who received at least 1 dose of study drug and had sufficient PK data to derive at least 1 PK parameter.

The PD Analysis Set is the group of subjects who received at least 1 dose of study drug and had sufficient PD data to derive at least 1 PD parameter.

The definition of these analysis sets is the same for both the Phase 1b and Phase 2 Treatment Periods; actual determination of these analysis sets will be made separately for each study period.

1.-8. (canceled)

9. A method of treating a human subject diagnosed with a Tauopathy, comprising administering intravenously an antibody that specifically binds Tau to the human subject, wherein the antibody is administered as a single dose of about 3 mg/kg to about 90 mg/kg.

10. The method according to claim 9, wherein the antibody is administered as a single dose of about 3 mg/kg, about 10 mg/kg, about 30 mg/kg, about 60 mg/kg, or about 90 mg/kg.

11. A method of treating a human subject diagnosed with a Tauopathy, comprising administering an antibody that specifically binds Tau to the human subject, wherein the antibody is administered as a dose of about 750 mg to about 4500 mg.

12. The method of claim 11, wherein the antibody is administered as a dose of about 750 mg, about 1500 mg, about 3000 mg, or about 4500 mg.

13. The method, wherein the dose is administered once every four weeks.

14. The method of claim 11, wherein the dose is administered intravenously.

15. The method of claim 11, wherein the Tauopathy is Alzheimer's disease, frontotemporal dementia, or progressive supranuclear palsy.

16. The method of claim 15, wherein the frontotemporal dementia is Pick's Disease.

17. The method of claim 15, wherein the Alzheimer's Disease is Dominantly Inherited Alzheimer's Disease or sporadic Alzheimer's Disease.

18.-28. (canceled)

29. The method of claim 11, wherein the anti-Tau antibody comprises a heavy chain and a light chain, wherein the heavy chain comprises three heavy chain complementarity determining regions (HCDRs) (HCDR1, HCDR2 and HCDR3) and the light chain comprises three light chain complementarity determining regions (LCDRs) (LCDR1, LCDR2 and LCDR3), and
wherein the HCDR1 comprises the amino acid sequence of SEQ ID NO: 7; the HCDR2 comprises the amino acid sequence of SEQ ID NO: 8; the HCDR3 comprises the amino acid sequence of SEQ ID NO: 9; the LCDR1 comprises the amino acid sequence of SEQ ID NO: 10; the LCDR2 comprises the amino acid sequence of SEQ ID NO: 11; and the LCDR3 comprises the amino acid sequence of SEQ ID NO: 12, wherein the CDRs are defined according to the method of Kabat; or
wherein the HCDR1 comprises the amino acid sequence of SEQ ID NO: 13; the HCDR2 comprises the amino acid sequence of SEQ ID NO: 14; the HCDR3 comprises the amino acid sequence of SEQ ID NO: 15; the LCDR1 comprises the amino acid sequence of SEQ ID NO: 16; the LCDR2 comprises the amino acid sequence of SEQ ID NO: 17; and the LCDR3 comprises the amino acid sequence of SEQ ID NO: 18, wherein the CDRs are defined according to the IMGT method.

30. (canceled)

31. The method of claim 11, wherein the anti-Tau antibody comprises a heavy chain variable domain (VH) comprising SEQ ID NO: 2 and a light chain variable domain (VL) comprising SEQ ID NO: 5.

32. The method of claim 11, wherein the anti-Tau antibody comprises a heavy chain comprising the amino acid sequence of SEQ ID NO: 1 and/or a light chain comprising the amino acid sequence of SEQ ID NO: 4.

33. (canceled)

34. The method of claim 9, wherein the Tauopathy is Alzheimer's disease, frontotemporal dementia, or progressive supranuclear palsy.

35. The method of claim 34, wherein the frontotemporal dementia is Pick's Disease.

36. The method of claim 34, wherein the Alzheimer's Disease is Dominantly Inherited Alzheimer's Disease or sporadic Alzheimer's Disease.

37. The method of claim 9, wherein the anti-Tau antibody comprises a heavy chain and a light chain, wherein the heavy chain comprises three heavy chain complementarity determining regions (HCDRs) (HCDR1, HCDR2 and HCDR3) and the light chain comprises three light chain complementarity determining regions (LCDRs) (LCDR1, LCDR2 and LCDR3), and
wherein the HCDR1 comprises the amino acid sequence of SEQ ID NO: 7; the HCDR2 comprises the amino acid sequence of SEQ ID NO: 8; the HCDR3 comprises the amino acid sequence of SEQ ID NO: 9; the LCDR1 comprises the amino acid sequence of SEQ ID NO: 10; the LCDR2 comprises the amino acid sequence of SEQ ID NO: 11; and the LCDR3 comprises the amino acid sequence of SEQ ID NO: 12, wherein the CDRs are defined according to the method of Kabat; or
wherein the HCDR1 comprises the amino acid sequence of SEQ ID NO: 13; the HCDR2 comprises the amino acid sequence of SEQ ID NO: 14; the HCDR3 comprises the amino acid sequence of SEQ ID NO: 15; the LCDR1 comprises the amino acid sequence of SEQ ID NO: 16; the LCDR2 comprises the amino acid sequence of SEQ ID NO: 17; and the LCDR3 comprises the amino acid sequence of SEQ ID NO: 18, wherein the CDRs are defined according to the IMGT method.

38. The method of claim 9, wherein the anti-Tau antibody comprises a heavy chain variable domain (VH) comprising SEQ ID NO: 2 and a light chain variable domain (VL) comprising SEQ ID NO: 5.

39. The method of claim 9, wherein the anti-Tau antibody comprises a heavy chain comprising the amino acid sequence of SEQ ID NO: 1 and/or a light chain comprising the amino acid sequence of SEQ ID NO: 4.


SEQUENCE LISTING (18 sequences)

SEQ ID NO: 1 - protein, 449 aa, Synthetic construct
EVQLLESGGGLVQPGGSLRLSCAASGYTFTTYWITWVRQAPGKGLEWVSDIYPGSSISNYNEKFKSRFTISVDNSKNTLYLQMNSLRAEDTAVYYCAREDGYDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 119 aa, Synthetic construct
EVQLLESGGGLVQPGGSLRLSCAASGYTFTTYWITWVRQAPGKGLEWVSDIYPGSSISNYNEKFKSRFTISVDNSKNTLYLQMNSLRAEDTAVYYCAREDGYDAWFAYWGQGTLVTVSS

SEQ ID NO: 3 - protein, 330 aa, Synthetic construct [sequence omitted]

SEQ ID NO: 4 - protein, 219 aa, Synthetic construct
DIQMTQSPSSLSASVGDRVTITCRSSQSILHSNGNTYLEWYQQKPGKAPKLLISKVSNRFSGVPSRFSGSGSGTDFTLTISSLQPEDFATYYCFQGSHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 112 aa, Synthetic construct
DIQMTQSPSSLSASVGDRVTITCRSSQSILHSNGNTYLEWYQQKPGKAPKLLISKVSNRFSGVPSRFSGSGSGTDFTLTISSLQPEDFATYYCFQGSHVPFTFGQGTKLEIK

SEQ ID NO: 6 - protein, 107 aa, Synthetic construct [sequence omitted]

SEQ ID NO: 7 - protein, 5 aa, Synthetic construct
TYWIT

SEQ ID NO: 8 - protein, 17 aa, Synthetic construct
DIYPGSSISNYNEKFKS

SEQ ID NO: 9 - protein, 10 aa, Synthetic construct
EDGYDAWFAY

SEQ ID NO: 10 - protein, 16 aa, Synthetic construct
RSSQSILHSNGNTYLE

SEQ ID NO: 11 - protein, 7 aa, Synthetic construct
KVSNRFS

SEQ ID NO: 12 - protein, 9 aa, Synthetic construct
FQGSHVPFT

SEQ ID NO: 13 - protein, 8 aa, Synthetic construct
GYTFTTYW

SEQ ID NO: 14 - protein, 8 aa, Synthetic construct
IYPGSSIS

SEQ ID NO: 15 - protein, 12 aa, Synthetic construct
AREDGYDAWFAY

SEQ ID NO: 16 - protein, 11 aa, Synthetic construct
QSILHSNGNTY

SEQ ID NO: 17 - skipped

SEQ ID NO: 18 - protein, 9 aa, Synthetic construct
FQGSHVPFT